(19) United States
(12) Patent Application Publication
(10) Pub. No.: US 2011/0281762 A1
(43) Pub. Date: Nov. 17, 2011

(54) High throughput screening for anaerobic microorganisms

(76) Inventors: Justin L. Sonnenburg, Redwood City, CA (US); Steven Kyle Higginbottom, Morgan Hill, CA (US); Erica D. Sonnenburg, Redwood City, CA (US)

(21) Appl. No.: 13/068,488

(22) Filed: May 11, 2011

Related U.S. Application Data
(60) Provisional application No. 61/395,656, filed on May 13, 2010.

(51) Int. Cl. C40B 30/06; C12Q 1/68; C12Q 1/02

(52) U.S. Cl. 506/10; 435/29; 435/6.12

(57) ABSTRACT

Methods and compositions are provided for the screening of candidate agents for their effect on the growth and colonization of hosts by anaerobic microorganisms, particularly microorganisms that comprise the gut microbiota of mammals. In some embodiments of the invention, candidate agents are screened for the ability to modulate growth of multiple microbes within a taxon, or functionally related microbes. In some embodiments of the invention, candidate agents are screened for the ability to modulate growth across a genus or functionally-defined group when the microorganisms are presented with a substrate of interest, which substrates include, without limitation, prebiotic compounds that promote expansion of divergent taxa within the microbiota, e.g. starch, fats, isoflavones, etc.

### BACKGROUND OF THE INVENTION

Pharmaceutical drug discovery, a multi-billion dollar industry, involves the identification and validation of therapeutic targets, as well as the identification and optimization of lead compounds. The explosion in numbers of potential new targets and chemical entities resulting from genomics and combinatorial chemistry approaches over the past few years has placed enormous pressure on screening programs. The rewards for identification of a useful drug are enormous, but the percentage of hits from any screening program is generally very low. Desirable compound screening methods solve this problem by both allowing for a high throughput so that many individual compounds can be tested; and by providing biologically relevant information so that there is a good correlation between the information generated by the screening assay and the pharmaceutical effectiveness of the compound.

Some of the more important features for pharmaceutical effectiveness are specificity for the targeted cell or disease, a lack of toxicity at relevant dosages, and specific activity of the compound against its molecular target. Therefore, one would like to have a method for screening compounds or libraries of compounds that allows simultaneous evaluation for the effect of a compound on different cellular pathways, where the assay predicts aspects of clinical relevance and potentially of future in vivo performance.

The normal microbiota of humans is exceedingly complex, and varies by individual depending on genetics, age, sex, stress, nutrition and diet of the individual. It has been calculated that a human adult houses about 10¹² bacteria on the skin, 10¹⁰ in the mouth, and 10¹⁴ in the gastrointestinal tract. The latter number is far in excess of the number of eucaryotic cells in all the tissues and organs which comprise a human.

The microbiota of the gut perform many metabolic activities, and influence the physiology of the host. Bacteria make up the majority of the gut microbiota, although it includes anaerobic members of archaea and eukarya. The majority of these microbes are obligate anaerobes, and a small percentage facultative anaerobes. Somewhere between 300 and 1000 different species live in the gut, however, it is probable that a smaller number of species dominate. Most belong to one of the genera: Bacteroides, Clostridium, Fusobacterium, Eubacterium, Ruminococcus, Peptococcus, Peptostreptococcus, and Bifidobacterium. Species from the genus Bacteroides alone constitute about 30% of all bacteria in the gut, suggesting that this genus is especially important in the functioning of the host.

The relationship between gut microbiota and humans is not merely commensal, but rather is a symbiotic relationship. The microorganisms perform a host of useful functions, such as fermenting unused energy substrates, influencing the development and homeostasis immune system, preventing growth of harmful, pathogenic bacteria, regulating the development of the gut, producing vitamins for the host, and impacting multiple aspects of host physiology that affect fat storage.

Without gut microbiota, the human body would be unable to utilize some of the undigested carbohydrates it consumes, because some members of gut microbiota have enzymes that human cells lack for breaking down certain polysaccharides. Carbohydrates that humans cannot digest without bacterial help include certain starches, fiber, oligosaccharides and sugars that are not digested and absorbed in the upper portion of the GI tract, e.g. lactose in the case of lactose intolerance and sugar alcohols, mucus produced by the gut, and proteins. Bacteria turn carbohydrates they ferment into short chain fatty acids, or SCFAs. These materials can be used by host cells, providing a major source of useful energy and nutrients for humans. SCFAs increase the gut's absorption of water, reduce counts of damaging bacteria, increase growth of human gut cells, and are also used for the growth of indigenous syntrophic bacteria. Proteolytic fermentation breaks down proteins like enzymes, proteinaceous components of dead host and bacterial cells, and collagen and elastin found in food, and can produce toxins and carcinogens in addition to SCFAs. Evidence also suggests that bacteria enhance host absorption and storage of lipids.

Bacteria are also implicated in preventing allergies. Studies on the gut microbiota of infants and young children have shown that those who have or later develop allergies have different compositions of gut microbiota from those without allergies, with higher chances of having the harmful species C. difficile and S. aureus and lower prevalence of Bacteroides and Bifidobacteria. Another indicator that bacteria in the microbiota help maintain immune system homeostasis is the epidemiology of Inflammatory Bowel Disease, or IBD, such as Crohn's Disease (CD). The incidence and prevalence of IBD is high in industrialized countries with a high standard of living and low in less economically developed countries, having increased in developed countries throughout the twentieth century. The disease is also linked to good hygiene in youth; lack of breastfeeding; and consumption of large amounts of sucrose and animal fat. Its incidence is inversely linked with poor sanitation during the first years of life and consumption of fruits, vegetables, and unprocessed foods. Also, the use of antibiotics, which kill native gut microbiota and harmful infectious pathogens alike, especially during childhood, is associated with inflammatory bowel disease.

Changing the numbers and species of gut microbiota can reduce the body's ability to ferment carbohydrates and metabolize bile acids and may cause diarrhea. Carbohydrates that are not broken down may absorb too much water and cause runny stools, or lack of SCFAs produced by gut microbiota could cause the diarrhea.

Various approaches have been suggested for the therapeutic exploitation of the commensal microbiota, including the use of pharmaceutical agents, live probiotic bacteria, probiotic-derived biologically active metabolites, prebiotics, synbiotics or genetically modified commensal bacteria. For example, prebiotics are dietary components that may help foster the growth of microorganisms in the gut. However, current agents are non-selective and fail to achieve the goal of altering the species composition of gut microbiota.

Methods that provide for reproducible, effective screening of compounds that affect the growth of one or more anaerobic microorganisms are of interest for many purposes, including development of clinically useful compounds. The present invention addresses this need.

### SUMMARY OF THE INVENTION

Methods and compositions are provided for the screening of candidate agents for their effect on the growth and colonization of hosts by anaerobic microorganisms, particularly microorganisms that comprise the gut microbiota of mammals. In one embodiment, methods and compositions are provided for novel high-throughput screening approaches. Such methods generally comprise contacting an anaerobic cell culture, which may comprise one or a plurality of species of anaerobic microorganisms, with a candidate agent of interest, and determining the effect of said agent on a parameter of cell growth, physiological status, and the like.

The use of highly controlled and reproducible conditions is of particular interest for the methods of the invention. In some embodiments of the invention, assays are performed using medium that has been formulated for improved stability. Stability is improved by identification of labile but necessary components in media, where the identified labile component(s) is then added to the medium shortly before culture of the microorganisms. In other embodiments, assays are performed where the composition of gases in the anaerobic chamber is optimized for the microorganism being cultured. In other embodiments of the invention, assays are performed where the microbial culture used for inoculation is selected to be synchronized with respect to physiological status. In other embodiments of the invention, assays are performed where media viscosity is adjusted to maintain cell buoyancy throughout growth.

In some embodiments of the invention; the assays are designed to minimize edge effects. Assay modifications to reduce edge effects include modifications to minimize media loss; pre-equilibrating media in the anaerobic environment; and pre-equilibrating media to the desired assay temperature.

In some embodiments of the invention, candidate agents are screened for the ability to modulate growth across a taxon, where a taxon may include, for example: multiple species in a genus, or strains in a species. The microorganisms may alternatively represent a functional group rather than evolutionarily-related group, in which certain genes or homologous proteins are shared between members of the group, e.g. from lateral gene transfer. In other variations of a “functional group,” microbes may not share genes but may share certain functions important to the microbe or host (e.g. metabolic pathways and functions, functions to alter the environment for preferential growth of the microbe, or interactions with the host or other microbes). In some embodiments of the invention, candidate agents are screened for the ability to modulate growth across the taxon of interest when the microorganisms are presented with a substrate of interest, which substrates include, without limitation, prebiotic compounds that promote expansion of divergent taxa within the microbiota, e.g. starch, fats, isoflavones, amino acids, etc.

In some cases, it is desirable to modulate the growth of one representative within a taxon. Therefore, in some embodiments, candidate agents are screened for the ability to modulate growth of a specific microbe, for example a specific species or specific strain within a species.

In some methods of the invention, an anaerobic culture of a strain derived from the microbiota is exposed both to a non-selective prebiotic compound, and to a candidate agent, usually a library of candidate agents, and the growth or substrate utilization of the microorganism is determined.

Species of interest include both those which would be targeted for inhibition (for example species known to play a causative role in or are correlated or assumed to be correlated with a specific pathological condition or activity) or those which would be targeted for enhanced growth (for example species known to play a beneficial role in health or are correlated or presumed to be correlated with a healthy function or combat of a pathological condition. Species of interest may also be identified as those microorganisms potentially problematic or beneficial in microbiota function and/or interaction with the host. Methods of identifying such microorganisms include screening for ability to utilize the substrate, such as an initial screening of growth with the prebiotic compound on a collection of microbiota-derived bacterial species to identify prebiotic-consuming species. Analysis of the taxa that preferentially expand within a mammalian microbiota when exposed to the prebiotic may utilize any suitable method of analysis, e.g. 16S rRNA-based enumeration. Such analysis may be performed in vitro or using in vivo models, e.g. gnotobiotic mice colonized with selective species or with a humanized microbiota, and the like.

In some embodiments, two or more related microbes are subjected to the screening method. In some embodiments, one type (i.e. taxa or other genetic or functional classification) of microbe is preferentially targeted over another. In other embodiments, two or more types of microbes are targeted over two or more other types. In one embodiment, the species may be selected based on sequence similarity at one or more loci of interest, which loci may include, without limitation, loci involved in utilization of the prebiotic compound. In some embodiments, the locus of interest is one or more polysaccharide utilization locus, or PUL, e.g. encoding SusC-like, SusD-like, and the like.

In other embodiments of the invention, prebioticindependent nutrient utilization pathways are screening targets for candidate agents, the inhibition of which negatively impact the taxon's abundance.

Efficacy of a candidate agent on the microbiota may be further assessed with an in vivo model, usually a non-human animal model. The animal model may have the feature of a gnotobiotic animal with known specific restricted microbial composition. In a specific embodiment, the gnotobiotic animal is “humanized” in that it contains microbes colonized directly from a human or representing a basic human-like microbial profile. A candidate agent is administered to the animal model, optionally in a combination with a prebiotic compound, and the effect on the distribution of species within the microbiota is determined, e.g. for short term and long term time points. For example, a feces sample may be tested for genetic diversity and population distribution using 16S rRNA or other genetic markers. Alternatively a biopsy sample may be obtained and assessed.

Small molecules or other compounds or components that may be used as drugs or supplements are identified using high-throughput cell based screening of anaerobic bacteria that that are indigenous to the human anatomical areas. Identification of such compounds provides lead compounds that can be developed for a new class of therapeutics: microbiota-targeted drugs or supplements. Additionally, this approach enables basic studies in which the microbiota can be rationally manipulated.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1. Bacteroides thetaiotaomicron (Bt) use of fructose-containing carbohydrates corresponds to induction of the polysaccharide utilization locus BT1757-1763 and BT1765. A. Genomic organization of Bt's Sus locus (top) and putative fructan utilization locus (bottom). Genes of similar function are coded by color; intervening unrelated genes are white; genes without corresponding homologs are grey. B. Gene expression patterns of differentially regulated susC and susD homologs from Bt grown in rich medium (TYG) at five time points from early log (3.5 h) to stationary phase (8.8 h) in duplicate. C. Growth curves of Bt in minimal medium containing indicated carbon source at 0.5% w/v. FOS, fructo-oligosaccharide. D. RNA abundance for genes relevant to fructan use in cells grown in different carbon sources, relative to growth in minimal medium plus glucose. Standard errors of expression levels from three biological replicate cultures are shown.

FIG. 2. BT1754 Hybrid two-component systems (HTCS) binds fructose and is required for growth on fructose-containing carbohydrates A. Growth curves of Bt-ΔBT1754 compared to wild type Bt (WT) and the complemented mutant (ΔBT1754::BT1754) on fructose-based carbon sources. B. Domain organization of BT1754. C. Interaction of the N-terminal periplasmic domain of BT1754 with fructose or levanbiose assessed by isothermal calorimetry, showing the raw heats of binding (upper panel) and integrated data (lower panel) fit to a single site binding model (fructose only). Values are 27 averages and SDs of at least three independent titrations.

FIG. 3. BT1760 encodes an extracellular endo-levanase required for Bt growth in levan A. Growth curves of Bt-ΔBT1760 compared to the complemented mutant (ΔBT1760::BT1760) in levan (top) or FOS (bottom panel). B. Thin layer chromatography (TLC) analysis of the products of levan digestion by the Bt GH32 enzymes, BT1760, BT1759, BT1765 and BT3082. Frc, fructose; L2, levanbiose; L3, levantriose; L4, levantetraose. C. Degradation of levan by Bt cells grown in minimal medium plus fructose. Error bars show the standard deviation (SDs) from three independent experiments.

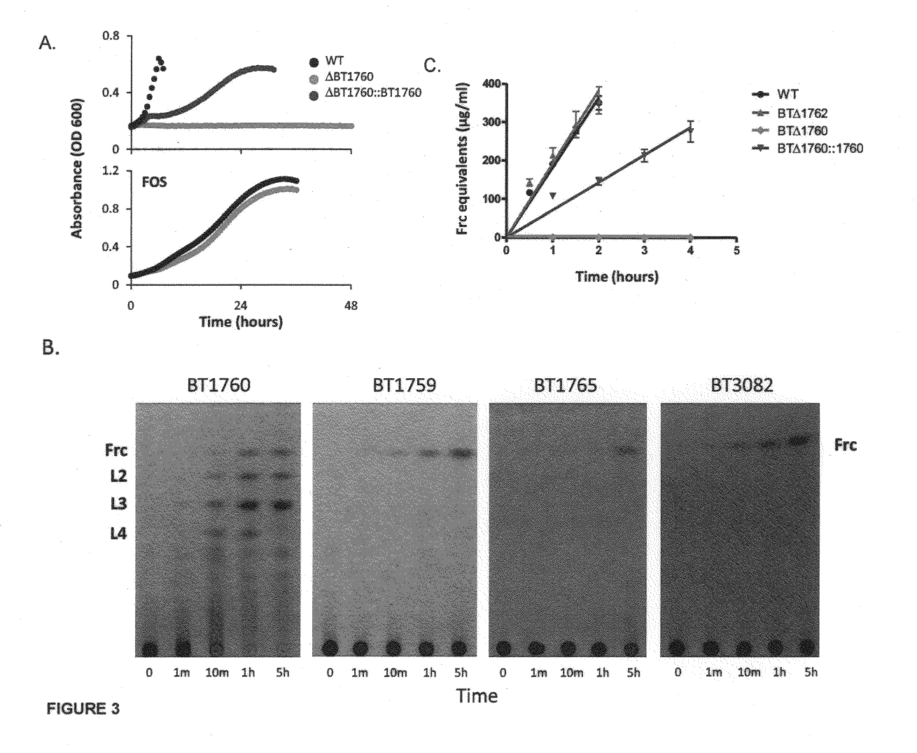

FIG. 4. The SusD-homolog encoded by BT1762 is required for efficient Bt utilization of levan and binds β2-6 but not β2-1 fructan A. Growth curves of wild type Bt, Bt-ΔBT1762, and Bt-ΔBT1762::BT1762 in levan (left) or FOS (right). B. Interaction of BT1762 with fructans as assessed by isothermal calorimetry. Levan binding data integrated and fit to a single site binding model (bottom left). Values are averages and SDs of at least three independent titrations.

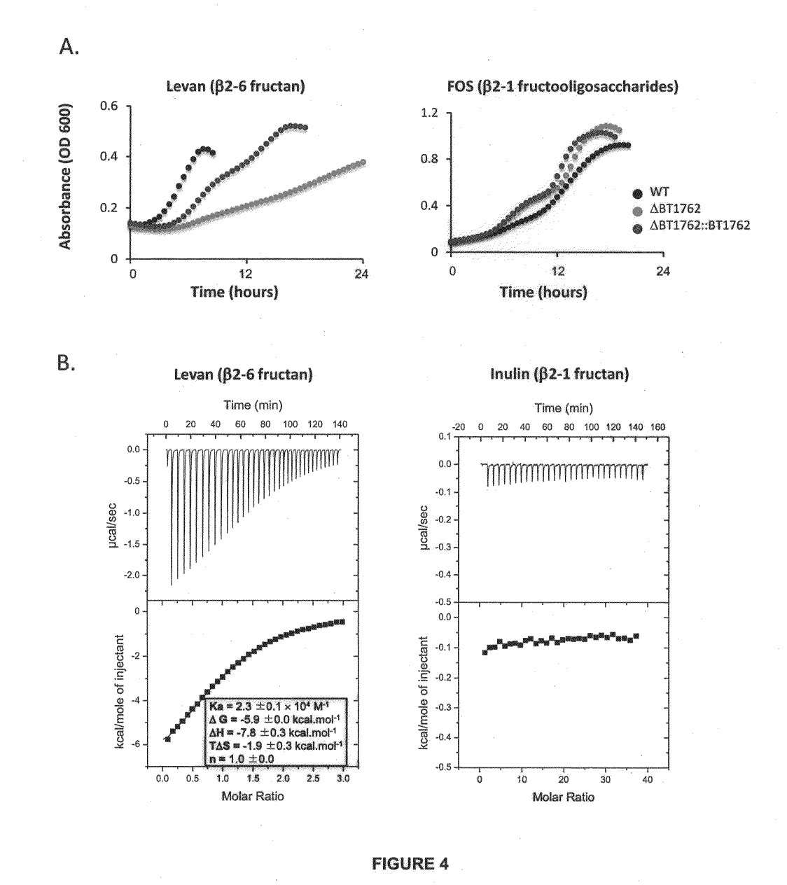

FIG. 5. Comparative genomic and functional analysis of fructan utilization among Bacteroides species. Fructan-utilization loci from Bacteroides species (left). Common predicted functions are color coded, intervening unrelated genes are white. PL19, polysaccharide lyase family 19; GH32, glycoside hydrolase family 32. Growth curves (right) of each Bacteroides species in fructosebased carbohydrates.

FIG. 6. Effect of dietary fructans on Bacteorides competition within the intestine A. Experimental design for in vivo experiments. GF, germ-free. B. Average relative fecal proportion (% total bacteria) of Bt and B. caccae at 4, 6, 14, and 21 days after colonization; n=7 mice. C. Average relative fecal proportion (% total bacteria) of Bt and B. vulgatus at 4, 6, 14, and 21 days after colonization; n=3 mice. D. Increase in proportion (%) of B. caccae over Bt from day 6 (1 day prior to diet change) to day 21 (14 days after diet change). All groups received a standard diet on days 1-7; type of diet and whether the mice received inulin in their water on days 7-21 is indicated; n=3-7 individually housed mice. E. Average relative fecal proportion (% total bacteria) of inulin-utilizing Bt(In+) and B. caccae at 4, 6, 14, and 21 days after colonization; n=7 individually housed mice.

FIG. 7. Uniform growth across 384-well plates and between plates for anaerobically grown microbes, is improved with novel tightly controlled conditions. Top panel shows optical density (OD) across two plates for which standard anaerobic growth conditions were used, and illustrates the high degree of growth variability. Bottom panel illustrates culture density for two plates in which uniform growth was achieved, by using innovations described herein. Reproducibility necessary for anaerobic cell based screening is achieved. Columns 23 and 24 were not inoculated and serve as negative controls in the assay.

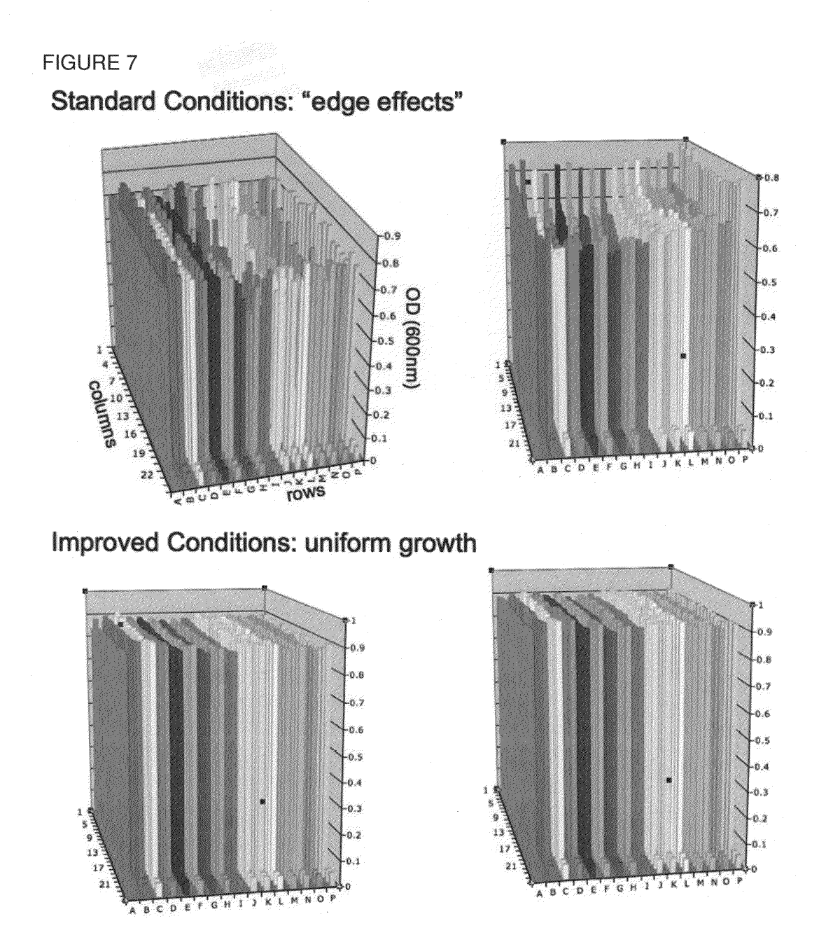

### DESCRIPTION OF THE SPECIFIC EMBODIMENTS

Methods and compositions are provided for the screening of candidate agents for their effect on the growth and colonization of hosts by anaerobic microorganisms, particularly microorganisms that comprise the gut microbiota of mammals. Such microorganisms include, without limitation, facultative anaerobic bacteria, obligate anaerobic bacteria, facultative anaerobic archaebacteria, obligate anaerobic archaebacteria, facultative eukaryotic microorganisms, and obligate eukaryotic microorganisms. Such methods generally comprise contacting an anaerobic cell culture, which may comprise one or a plurality of species of anaerobic microorganisms, with a candidate agent of interest, and determining the effect of said agent on a parameter of cell growth, physiological status, and the like. Assays may be performed utilizing the improved culture and screening techniques described herein. In one embodiment, the invention provides for a high-throughput screening approach. In some embodiments, candidate agents are screened for the ability to modulate growth of multiple members of a given taxon, optionally in combination with growth on a prebiotic substrate.

The microbiome offers a set of species and genes that are amenable to manipulation, providing therapeutic options. Therapeutic strategies include, without limitation, methods of altering the composition of an individual's microbiota to delay or prevent the onset of inflammatory bowel diseases or colon cancer, optimize dietary caloric extraction based on an individual's nutritional status and genotype, provide individuals with optimal and managed microbiotas to decrease the incidence of infectious diseases, and reverse the trend of increasing allergic and auto-immune disorders associated with hyper-hygienic industrialized countries. Intentional modulation of the composition or function of the microbiota is likely to be relevant in correcting or preventing numerous health-related issues.

The microbiota provides a wealth of novel drug targets with unprecedented exposure to oral therapeutics. These new targets are easily accessed since they reside on the surfaces of our bodies (e.g., within the digestive tract), therefore no host membranes or barriers need to be crossed for direct interaction with orally administered drugs. The identification of promising lead compounds need not be hampered by the conventional considerations of drug-like properties, e.g. bioavailability, half-life, etc. Such considerations may be irrelevant for compounds that target unique cell surface proteins of components of the microbiota and do not need to be absorbed by the host. Retention of compounds within the lumen of the gastrointestinal tract is also likely to greatly reduce drug side-effects.

The methods of the invention relate to the identification of strategy will permit the expansion of taxa that are indigenous to the microbiota, and thereby circumvent safety, regulatory and technical issues associated with the alternative of oral administration of non-food-based “probiotics”. Additionally, the inhibitor-based strategy permits eradication or reduction in the levels of unwanted taxa, a distinct advantage over the use of probiotics and a task that will be of prime importance as disease-associated microbes within the microbiota are identified.

Screening strategies include the identification of agents that modulate composition or function of the microbiota. Agents may be screened for impacting one or more aspects of microbial cell biology within an anaerobic environment, e.g., ability to activate or inhibit the growth, which modulation may be in absolute terms or in terms of growth relative to other organisms, e.g. organisms present in the microbiota of an organism of interest; modulate aspects of cel physiology, such as cell shape or motility or permeability; antagonize or agonize a signaling pathway; or elicit or inhibit other aspects of microbial cell function. The modulation of the microbe may be accomplished in the presence of a medium or environment normally provided to the anaerobic microorganism, or may be a medium or environment that is supplemented with a prebiotic or other compounds of interest. Identification of candidate agents generally involves contacting a microorganism of interest under anaerobic growth conditions with a candidate agent, and determining the growth or phenotype of the organism in the presence of the agent. Such contacting may be accomplished in the presence of a prebiotic agent of interest. Other endpoints of assay assessment such as colorimetric indicators that can detect changes in redox state or pH of the growth medium that occur during fermentation may be implemented as necessary to optimize the assay (e.g., maximize dynamic range), in addition to measuring turbidity, colony counts, etc.

Flexible multiplex screening assays are provided for the screening and biological activity classification of candidate agents. Where a candidate agent is brought into contact with one or a plurality of anaerobic microorganisms, the assay optionally further comprises additional screening variations. In some embodiments, the effect of an agent on alteration of growth environment is tested with a panel of microorganisms, i.e. two or more, related species or strains, which species may be across a taxon; across functionally related species; etc. Alternatively or in combination with a panel of microorganisms, screening can be performed in the presence of multiple growth environments, including without limitation one or more growth environments comprising the presence of one or more prebiotic compounds. The effect of the agent and/or altered growth environment may be assessed by monitoring cell growth, cell phenotype, substrate utilization, and the like. The term “assay combination” may be used herein to refer to the sum of components utilized in an assay mixture, for example a combination of microbe, candidate agent and prebiotic compound, a plurality of microbes and a candidate agent, etc. A panel of assay combinations may be provided, for example a panel comprising multiple candidate agents, where each assay mixture differs only in the candidate agent; a panel comprising multiple strains or species across a taxon, where each assay mixture differs only in the microorganism; a panel comprising multiple prebiotic compounds; and the like.

The term “environment,” or “culture condition” encompasses cells, prebiotics, media, time and temperature. Environments may also include drugs and other compounds, particular atmospheric conditions, pH, salt composition, minerals, etc. More complex environments may be useful and incorporated into the assay, such environments including conditions simulating the intestinal milieu, inclusion of host cells such as epithelial or immune cell types or derived factors, combinations of commensal or pathogenic microbes constituting a generalizable or specific microbiota, or other factors that might simulate the natural environment of the targeted microbial population in either a healthy or altered (i.e. disease-specific) state. The conditions will be controlled and the resulting dataset of results will reflect the similarities and differences between each of the assay combinations involving a different environment or culture condition. Culture of cells is typically performed in a sterile anaerobic environment, for example, at 37° C. in an incubator.

### DEFINITIONS

Microbiota. As used herein, the term microbiota refers to the set of microorganisms present within an individual, usually an individual mammal and more usually a human individual. Of particular interest is the microbiota of the gut. While the microbiota may include pathogenic species, in general the term references those commensal organisms found in the absence of disease. The gut microbiota of adult humans is primarily composed of obligate anaerobic bacteria.

In a healthy animal, while the internal tissues, e.g. brain, muscle, etc., are normally presumed to be free of microorganisms, the surface tissues, i.e., skin and mucous membranes, are constantly in contact with environmental organisms and become readily colonized by various microbial species. The mixture of organisms known or presumed to be found in humans at any anatomical site is referred to as the “indigenous microbiota”.

In humans, there are differences in the composition of the microbiota which are influenced by age, diet, cultural conditions, and the use of antibiotics. The microbiota of the large intestine (colon) is qualitatively similar to that found in feces. Populations of bacteria in the colon reach levels of 10¹¹/ml feces. The intestinal microbiota of humans is dominated by species found within two bacterial phyla: members of the Bacteroidetes and Firmicutes make up >90% of the bacterial population. Actinobacteria (e.g., members of the Bifidobacterium genus) and Proteobacteria among several other phyla are less prominently represented. Significant numbers of anaerobic methanogens (up to 10¹⁰/gm) may reside in the colon of humans. Common species of interest include prominent or less abundant members of this community, and may comprise, without limitation, Bacteroides thetaiotaomicron; Bacteroides caccae; Bacteroides fragilis; Bacteroides melaminogenicus; Bacteroides oralis; Bacteroides uniformis; Lactobacillus; Clostridium perfringens; Clostridium septicum; Clostridium tetani; Bifidobacterium bifidum; Staphylococcus aureus; Enterococcus faecalis; Escherichia coli; Salmonella enteritidis; Klebsiella sp.; Enterobacter sp.; Proteus mirabilis; Pseudomonas aeruginosa; Peptostreptococcus sp.; Peptococcus sp., Faecalibacterium sp,; Roseburia sp.; Ruminococcus sp.; Dorea sp.; Alistipes sp.; etc.

The composition of the microbiota of the gastrointestinal tract varies longitudinally along the tract (along the cephalocaudal axis) and transversely across the tract (with increasing distance from the mucosa). There is frequently a very close association between specific bacteria in the intestinal ecosystem and specific gut tissues or cells (evidence of tissue tropism and specific adherence). Gram-positive bacteria, such as the streptococci and lactobacilli, are thought to adhere to the gastrointestinal epithelium using polysaccharide capsules or cell wall teichoic acids to attach to specific receptors on the epithelial cells. Members of the segmented filamentous bacteria (SFBs) adhere to intestinal epithelium using a specialized structure on the cell surface known as a holdfast. Gram-negative bacteria such as the enterics may attach by means of specific fimbriae which bind to glycoproteins on the epithelial cell surface.

In addition, cells including bacterial cells that have been genetically altered with recombinant genes or by gene deletion or mutation, to provide a gain or loss of genetic function, may be utilized with the invention. Methods for generating genetically modified cells are known in the art. The genetic alteration may be a knock-out, usually where homologous recombination results in a deletion that knocks out expression of a targeted gene; or a knock-in, where a genetic sequence not normally present in the cell is stably introduced.

A variety of methods may be used in the present invention to achieve a knock-out, including site-specific recombination, expression of dominant negative mutations, and the like. Knockouts have a partial or complete loss of function in the endogenous gene in the case of gene targeting. Preferably expression of the targeted gene product is undetectable or insignificant in the cells being analyzed. This may be achieved by introduction of a disruption of the coding sequence, e.g. insertion of one or more stop codons, insertion of a DNA fragment, etc., deletion of coding sequence, substitution of stop codons for coding sequence, etc. In some cases the introduced sequences along with native regions of the genome are ultimately deleted from the genome, leaving a net change to the native sequence.

The introduction of the genetic agent results in an alteration of the total genetic composition of the cell. Genetic agents such as DNA can result in an experimentally introduced change in the genome of a cell, generally through the integration of the sequence into a chromosome. Genetic changes can also be transient, where the exogenous sequence is not integrated but is maintained as an episomal agent. Genetic agents, such as antisense oligonucleotides, can also affect the expression of proteins without changing the cell's genotype, by interfering with the transcription or translation of mRNA. The effect of a genetic agent is to increase or decrease expression of one or more gene products in the cell.

Introduction of an expression vector encoding a polypeptide can be used to express the encoded product in cells lacking the sequence, or to over-express the product. Various promoters can be used that are constitutive or subject to external regulation, where in the latter situation, one can turn on or off the transcription of a gene. These coding sequences may include full-length coding sequences, fragments derived therefrom, or chimeras that combine a naturally occurring sequence with functional or structural domains of other coding sequences. Alternatively, the introduced sequence may encode an anti-sense sequence; be an anti-sense oligonucleotide; encode a dominant negative mutation, or dominant or constitutively active mutations of native sequences; altered regulatory sequences, etc.

Methods that are well known to those skilled in the art can be used to construct expression vectors containing coding sequences and appropriate transcriptional and translational control signals for increased expression of an exogenous gene introduced into a cell. These methods include, for example, in vitro recombinant DNA techniques, synthetic techniques, and in vivo genetic recombination. Alternatively, RNA capable of encoding gene product sequences may be chemically synthesized using, for example, synthesizers. See, for example, the techniques described in “Oligonucleotide Synthesis”, 1984, Gait, M. J. ed., IRL Press, Oxford.

For various purposes of the invention it is desirable to compare the effect of an agent on multiple related microbes. For such purposes a locus of interest may be defined in the species, where the locus is usually involved in utilization of the prebiotic compound, e.g. starch utilization operon. For the purposes of the invention, related mirobes generally share a high degree of sequence similarity at the locus of interest however in rare cases, overall sequence similarity may be low, but conservation of key functional amino acids (e.g., catalytic residues, substrate binding pockets, etc.) may suffice. The bioinformatic identification of genomic regions (and encoded functions, metabolic pathway, regulatory pathways, etc.) that are conserved within a monophyletic clade and define a given taxon at a given level of phylogenetic resolution serves as an additional method to identify optimal screening targets. Identified inhibitors would act specifically on the taxon of interest. Alternatively, the identification of genes critical to a function of interest may be targeted for inhibition irrespective of phylogenetic relationship of microbes harboring these genes (e.g., if inhibition of a functionally related group, rather than a monophyletic group is desired).

Prebiotic compounds. As used herein the term “prebiotic” refers to food ingredients that are not digested by the mammal that ingests them, but which are a substrate for the growth or activity of the microbiota, particularly the gut microbiota. Many prebiotics are carbohydrates, e.g. polysaccharides and oligosaccharides, but the definition does not preclude non-carbohydrates. The most prevalent forms of prebiotics are nutritionally classed as soluble fiber. Prebiotics may provide for changes in the composition and/or activity of the gastrointestinal microbiota, however it is shown herein that prebiotics are typically non-selective and are coveted substrates for multiple taxa of the microbiome. See Gibson and Roberfroid Dietary modulation of the human colonic microbiota: introducing the concept of prebiotics. J. Nutr. 1995 June; 125(6):1401-12, herein incorporated by reference.

Prebiotics of interest can include inulin, fructooligosaccharides (FOS), xylooligosaccharides (XOS), polydextrose, mannooligosaccharides, tagatose, galactooligosaccharides (GOS), gum guar, gum Arabic, amylose, amylopectin, xylan, pectin, and the like.

Candidate Agents. Candidate agents of interest are biologically active agents that encompass numerous chemical classes, primarily organic molecules (which may include organometallic molecules), inorganic molecules, nucleotide sequences, etc. The agents additionally include biological components of any macromolecular class, including proteins, peptides, nucleic acids, carbohydrates, or lipids, or combinations thereof (e.g. glycoporteins). An important aspect of the invention is to evaluate candidate drugs, select therapeutic antibodies and protein-based therapeutics, or other agents with potential therapeutic value, with preferred biological response functions. Candidate agents generally comprise functional groups necessary for structural interaction with biological macromolecules, particularly hydrogen bonding, and typically include an amine, carbonyl, hydroxyl or carboxyl group, and frequently two or more of the functional chemical groups. The candidate agents may comprise cyclical carbon or heterocyclic structures and/or aromatic or polyaromatic structures substituted with one or more of the above functional groups. Candidate agents may also be found among biomolecules, including peptides, polynucleotides, saccharides, fatty acids, steroids, purines, pyrimidines, derivatives, structural analogs or combinations thereof.

Compounds, including candidate agents, are obtained from a wide variety of sources including libraries of synthetic or natural compounds. For example, numerous means are available for random and directed synthesis of a wide variety of organic compounds, including biomolecules, including expression of randomized oligonucleotides and oligopeptides. Additionally, agents for screening may be derived from foods, cosmetics, or other products that are already consumed or used by humans. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

The term “samples” also includes the fluids described above to which additional components have been added, for example components that affect the ionic strength, pH, total protein concentration, etc. In addition, the samples may be treated to achieve at least partial fractionation or concentration. Biological samples may be stored if care is taken to reduce degradation of the compound, e.g. under nitrogen, frozen, or a combination thereof. The volume of sample used is sufficient to allow for measurable detection, usually from about 0.1 μl to 1 ml of a biological sample is sufficient.

Agents are screened for biological activity by adding the agent to at least one and usually a plurality of assay combinations to form a panel of assay combinations, usually in conjunction with assay combinations lacking the agent. The change in parameter readout in response to the agent is measured, desirably normalized, and the resulting data may then be evaluated by comparison to reference samples. The reference samples may include basal readouts in the presence and absence of the candidate agent, absence of the prebiotic, readouts obtained with other agents, which may or may not include known inhibitors of known pathways, etc. Agents of interest for analysis include any biologically active molecule with the capability of modulating, directly or indirectly, a cell of interest.

The agents are conveniently added in solution, or readily soluble form, to the medium of cells in culture. The agents may be added in a flow-through system, as a stream, intermittent or continuous, or alternatively, adding a bolus of the compound, singly or incrementally, to an otherwise static solution. As described in the inventive concepts contained herein, the system will generally be maintained in an oxygen-free environment. In a flow-through system, two fluids are used, where one is a solution compatible with microbial physiology, and the other is the same solution with the test compound added. The first fluid is passed over the cells, followed by the second. In a single solution method, a bolus of the test compound is added to the volume of medium surrounding the cells. The overall concentrations of the components of the culture medium should not change significantly with the addition of the bolus, or between the two solutions in a flow through method.

Preferred agent formulations do not include additional components, such as preservatives, that may have a significant effect on the overall formulation. Thus preferred formulations consist essentially of a biologically active compound and a physiologically acceptable carrier, e.g. water, ethanol, DMSO, etc. However, if a compound is liquid without a solvent, the formulation may consist essentially of the compound itself.

A plurality of assays may be run in parallel with different agent concentrations to obtain a differential response to the various concentrations. As known in the art, determining the effective concentration of an agent typically uses a range of concentrations resulting from 1:10, or other log scale, dilutions. The concentrations may be further refined with a second series of dilutions, if necessary. Typically, one of these concentrations serves as a negative control, i.e. at zero concentration or below the level of detection of the agent or at or below the concentration of agent that does not give a detectable change in the phenotype.

### METHODS OF THE INVENTION

Methods and compositions are provided for the screening, and specifically high through-put screening, of candidate agents for their effect on the growth and colonization of hosts by anaerobic microorganisms. Such methods generally comprise contacting an anaerobic cell culture, which may comprise one or a plurality of species of anaerobic microorganisms, with a candidate agent of interest, and determining the effect of said agent on a parameter of cell growth, physiological status, and the like. Assays may be constructed and performed utilizing the improved culture and screening techniques described herein.

In some methods of the invention, an anaerobic culture of a species within the microbiota is exposed both to a non-selective prebiotic compound, and to a candidate agent, usually a library of candidate agents, which may be referred to as an assay combination, and the growth, phenotype, physiological response, or substrate utilization of the microorganism is determined. In some methods of the invention, an anaerobic culture of a species within the microbiota is exposed to a candidate agent, usually a library of candidate agents, which may be referred to as an assay combination, and the growth, phenotype or substrate utilization of the microorganism is determined.

Where a prebiotic compound is included in the assay, the selection of a prebiotic compound may utilize various methods, with a preference given for prebiotic compounds that lack specificity and promote expansion of divergent taxa within the microbiota. Most currently used prebiotic compounds are appropriate for screening consideration. Galactooligosaccharides increase the prevalence not only of Bifidobacterium species, but also members of the Clostridium perfringens-histolyticum subgroup of the Firmicutes. Furthermore, studies focused on identifying new polysaccharide prebiotic compounds using batch culture fermentation of gut bacteria has demonstrated that arabinan stimulates the growth of Bifidobacterium and Bacteroides species.

The choice of microbial species for screening may be guided by published studies and experimental data. For example, with respect to the major classes of prebiotics, sufficient published data (including human trials, studies in animal models, and culture-based screens that identify species adept at utilization of specific prebiotics) exist to identify taxa that are candidates for screening. Candidates could be species known to grow well on a specific prebiotic (where one would target inhibition of this consumption for pathogens, and enhancement of this consumption for beneficials), or a specific species that does not grow well on a given probiotic with the goal of enhancing that preferential growth.

Where there is insufficient data to identify suitable taxa for a given prebiotic compound, a prebiotic growth-screen may be conducted on a collection of microbiota-derived bacterial species to identify prebiotic-consuming species in vivo or in vitro. For in vivo screening, the preferential expansion of taxa within a mammalian microbiota when exposed to the prebiotic, may utilize bacterial enumeration studies, e.g. in gnotobiotic mice that harbor a humanized microbiota and are fed a prebiotic-supplemented diet. Genera of particular interest include Bacteroides and those within the Firmicutes phylum. Preferably multiple species or strains within the taxon of interest are screened to identify compounds with the best potential for a taxon-wide spectrum of inhibition. The combination of inhibitors for nutrient utilization pathways of the Bacteorides and the Firmicutes will not only allow for these populations to be held in check during prebiotic treatment, but will provide the tools necessary to determine how deviation of microbiota composition influences host biology. Thus, the methods and results of the invention are particularly useful for pharmacological uses as well as for development of research tools and mechanistic information critical to the microbiota and host health and disease.

Due to the cell-based nature of the screen, specific gene products will not be intentionally targeted, however the identification of relevant nutrient utilization systems ensures that good potential targets do exist and are conserved within the taxon of interest. Genes coding for nutrient utilization machinery can be identified using transcriptional profiling or transposon screens conducted on a proxy representative of the taxon in defined medium containing the prebiotic as the sole fermentable carbohydrate. The induction of microbial nutrient utilization systems in the presence of their cognate substrate has been widely documented, and so that transcriptional profiling can be used to identify relevant genes when transposon mutagenesis is not possible.

Conservation of the candidate nutrient utilization genes within a taxon may be determined using comparative genomic analysis of available microbiota-derived genomes. Targets of interest may include systems that show conservation within a taxon that mirrors the ability to utilize the prebiotic, or that mirrors the species that expand in prebiotic trials. The data indicate that inhibition of a protein encoded within a single polysaccharide utilization locus can ablate utilization of the polysaccharide in multiple related species.

Identification of candidate agents that inhibit prebiotic utilization machinery utilizes the screening methods described herein. In some embodiments the growth of the microorganism may be sufficient. In such embodiments the agent is typically brought into contact with the species or multiple species of interest in the absence and presence of the prebiotic of interest. The control situation where growth is on a substrate other than the prebiotic provides a control against non-specific growth inhibition. Colorimetric indicators that can detect changes in redox state or pH of the growth medium that occur during fermentation may be implemented to maximize the dynamic range of the assay, in addition to measuring turbidity, colony counts, etc.

In other embodiments a gain-of-function strategy for screening may eliminate false positives caused by non-specific growth inhibitors and will permit moving to a sensitive and efficient screens. The gain-of-function screening uses a reporter gene linked to a nutrient utilization system of a low priority substrate. Screening is conducted in the presence of the prebiotic of interest and the less coveted substrate and therefore the reporter is constitutively repressed, and will only be expressed when prebiotic utilization is inhibited. For example, screening for B. theta fructan, i.e. levan, FOS, inulin, etc. utilization inhibitors can be conducted in the presence of a fructan and mannan, a polysaccharide comprised of mannose residues that is a low priority substrate for B. theta. In the presence of fructan, B. theta does not express its mannan utilization machinery, unless the fructan pathway is blocked. Utilization of mannan and expression of a reporter linked to the mannan utilizations locus, is indicative of successful inhibition of fructan utilization. Multiple reporter systems are compatible with anaerobic bacteria including the widely used β-glucuronidase-based system (GUS). While green fluorescent protein requires oxygen for fluorescence, B. theta can manufacture the protein in an anaerobic environment, and fluorescence can be detected upon exposure to oxygen. A recently developed reporter that fluoresces in the absence of oxygen permits fluorescent monitoring during anaerobic growth. Reporter strategies may be compatible with non-genetically manipulatable organisms if an endogenous reporter exists. For example, in the gain-of-function screen described above, the induction of the low priority mannan utilization locus results in B. theta's production of a secreted α-mannosidase whose activity can be detected by adding a mannoside-based reporter substrate (4-nitrophenyl- or 4-methylumbelliferyl-mannoside) to the medium. Therefore, in the absence of genetic manipulation colorimetric or fluorescent signal will coincide with inhibition of the FOS utilization system. Species not amenable to genetic manipulation and in which an endogenous reporter cannot be identified will be screened using the growth inhibition screen described previously.

### Assay Conditions and Devices

An important aspect of cell based screening of anaerobic microbes is conducting assays using highly controlled and reproducible conditions. A number of innovations related to anaerobic culturing methodology are described below, each of which singly or in combination can contribute to the reproducibility and utility of the assays.

In some embodiments of the inventions, assays are conducted in a medium that has been optimized for stability over time. It is desirable to conduct an assay in a medium in which the concentration of components does not vary between assays. However, certain components required for microbial growth can be labile. Components can be identified that contribute to variability in their susceptibility to degradation, precipitation, or other alterations in chemical or physical properties that occur over time. Such components, once identified, can be suitably stored in a manner that limits such alterations in properties, and added to the medium immediately prior to initiation of culture. For example, cysteine is identified herein as a component of Bacteroides minimal, defined media that is necessary for growth, and labile over time. In some aspects of the invention, cysteine is stored frozen in aliquots in concentrated form (e.g., 100× final concentration), e.g. −20° C., −70° C., −80°, etc. Medium is formulated without cysteine, and the cysteine concentrate is then added to the correct concentration after being thawed from frozen stock solution just prior to inoculation.

The concentration of anaerobic gases can be critical to the growth of the organisms of interest. In some embodiments of the invention, assays are performed where the relative concentration of gases has been optimized, e.g. by performing a growth curve of the organism of interest in the presence of varying gas concentrations to determine the specific requirements for growth. For example, CO₂ must be present for optimal Bacteroides growth. In addition, a concentration of H₂ is desirably maintained at a reproducible and minimum threshold level to obtain reproducible results, usually at least about 2%, at least about 3%, at least about 4% and not more than about 5% prior to assay initiation. If a catalyst is used to scrub out residual oxygen from the environment, hydrogen levels may decrease upon catalyst enabled reaction with oxygen, and thus the concentration should be corrected.

Assay initiation is preferably performed with cells of similar physiological status. When microbial cells are grown in batch culture, they typically undergo three phases during the ensuing culture: lag phase, growth phase, and stationary phase. It has been found that these phases of culturing can be highly variable between screens. In some embodiments of the invention, this variability is minimized by ensuring that the culture used to inoculate each screen consists of cells of a reproducible physiological status, e.g. having undergone the same number of doublings in the preceding culture, are in the same growth phase, etc. In one embodiment, reproducibility can be achieved by strictly adhering to a specific aliquot-to-medium volume ratio for starting the batch culture that is used to inoculate cultures for the assay, where the aliquots are glycerol stock aliquots of a given strain, which are stored frozen, e.g. −70°, −80° C., etc. If subculturing into the screening plates is performed at the same timepoint after inoculation for each screen, then the physiological status of the cells is reproducible and the ensuing growth within the screen is uniform.

The relative proximity of wells within each high-density plate, e.g. 96 well plate, 384 well plate, etc. to the outer edges of the plate can differentially impact the parameters, e.g., lag duration, growth rate, maximum density, of cell growth. In some embodiments of the invention, such “edge effects” are minimized within an anaerobic environment by controlling aspects of the environment that may vary across plates. Variables are identified herein that are contributors to edge effects, and methods are provided to minimize the impact of these variables on the assays.

Loss of media due to evaporation occurs most rapidly within the wells nearest the edge of the plate, which influences cell growth. Thus, in some embodiments of the invention the assays are conducted with a device that minimizes media loss. Such methods include, without limitation, conducting assays within humidified sub-chambers; conducting assays in plates that have been sealed with gas permeable membranes; conducting assays in plates having an organic phase overlay, e.g., mineral oil; and the like.

The rate at which a given well becomes anaerobic is related to its proximity to the edge of the plate, which also produces edge effects. Therefore, pre-equilibrating media within the high-density plates in the anaerobic environment prior to inoculation enhances uniform growth. The rate at which a given well reaches optimal growth temperature of 37° C., which occurs after being placed into an incubator, is also related to its proximity to the edge of the plate. Therefore, pre-equilibrating media within the plate to 37° C. prior to inoculation enhances uniform growth across the plate, e.g. for at least about 4 hours, at least about 12 hours, at least about 16 h generally provides a sufficient period of equilibration.

Bacteria that settle over the course of growth may reduce uniform growth and interfere with accurate density readings. Agitation or shaking of cells during growth and or prior to reading can circumvent these problems, but can be cumbersome and lack reproducibility on a large scale. In some embodiments of the invention, assays are performed where media viscosity is increased relative to liquid medium by the addition of a thickening agents, for example by formulating media with glycerol at a concentration of at least about 1% and not more than about 10%; by formulating media with agar at a concentration of at least 0.1% and not more than about 2.5%.

A dataset may comprise growth parameter results from a panel of assay combinations. Datasets may include not only the information that has been developed with the study, but also information that has been previously developed under comparable conditions. For example a panel may be used that is comprised of an assay combination that provides for a prebiotic and candidate agent of interest, where the panel provides for a plurality of agents tested in these conditions.

Desirably, a panel will comprise at least one assay combination that represents a basal or normal environment as a control for non-specific growth inhibition. The panel will desirably include multiple, usually related, species for comparison. In one embodiment, the panel of cells and culture conditions includes variants of representative culture condition(s), where single specific changes are made in order to expand the dataset, e.g. by providing combinatorial subsets of prebiotics, provision of known agents in the culture medium, utilizing cell variants comprising targeted genetic changes, etc. Various methods can be utilized for quantifying growth parameters.

A comparison of a dataset obtained from a test compound, and a reference dataset is accomplished by the use of suitable deduction protocols, systems, statistical comparisons, etc. Preferably, the dataset is compared with a database of reference datasets. Similarity to reference datasets can provide an initial indication of the cellular pathways targeted or altered by the prebiotic, test stimulus or agent.

A database of reference datasets can be compiled. These databases may include reference datasets from panels that include known agents or combinations of agents that target specific pathways, as well as references from the analysis of cells treated under environmental conditions in which single or multiple environmental conditions or parameters are removed or specifically altered. Reference datasets may also be generated from panels containing cells with genetic constructs that selectively target or modulate specific cellular pathways. In this way, a database is developed that can reveal the contributions of individual pathways to a complex response.

The readout may be a mean, average, median or the variance or other statistically or mathematically derived value associated with the measurement. The parameter readout information may be further refined by direct comparison with the corresponding reference readout. The absolute values obtained for each parameter under identical conditions will display a variability that is inherent in live biological systems and also reflects individual cellular variability as well as the variability inherent between individuals.

Demonstration of in vivo efficacy of the small molecule inhibitor in the mouse model will be accompanied by characterization of host responses to the change in microbiota composition. Additional tests may be dictated by the existing data regarding host health and disease status related to the prebiotic and bacterial species being studied. In diseases where therapeutic potential is supported, the inhibitor that allowed a basic question to be addressed in an animal model will serve as a candidate for drug development.

For convenience, the systems of the subject invention may be provided in kits. The kits may include one or more of appropriate prebiotics, inocula of anaerobic microbial species, which may be frozen, refrigerated or treated in some other manner to maintain viability, reagents for measuring growth parameters, and software. The software will receive the results and create a dataset and can include data from other assay combinations for comparison. The software can also normalize the results with the results from basal cultures, related or unrelated species, etc.

Kits may be provided comprising the agents identified by the screening methods of the invention, for example comprising a prebiotic and agent to be consumed for a fixed or indefinite period of time, to alter microbiota composition and/or function.

### EXPERIMENTAL

### Example 1

### Specificity of Polysaccharide Use in Intestinal Bacteroides Species Determines Diet-Induced Microbiota Alterations

The intestinal microbiota impacts many facets of human health and is associated with human diseases. Diet impacts microbiota composition, yet mechanisms that link dietary changes to microbiota alterations remain ill-defined. Here we elucidate the basis of Bacteroides proliferation in response to fructans, a class of fructose-based dietary polysaccharides. Structural and genetic analysis disclosed a fructose-binding, hybrid-two-component signaling sensor that controls the fructan utilization locus in Bacteroides thetaiotaomicron. Gene content of this locus differs among Bacteroides species and dictates the specificity and breadth of utilizable fructans. BT1760, an extracellular β-6 endo-fructanase, distinguishes B. thetaiotaomicron genetically and functionally, and enables the use of the β2-6-linked fructan levan. The genetic and functional differences between Bacteroides species are predictive of in vivo competitiveness in the presence of dietary fructans. Genes that differentiate function serve as potential biomarkers in microbiomic datasets to enable rational manipulation of the microbiota via diet.

Many complex plant polysaccharides in the human diet are resistant to host-mediated degradation due to either insolubility or lack of human-encoded hydrolytic enzymes. These carbohydrates are not absorbed in the upper gastrointestinal tract and serve as a major source of carbon and energy for the distal gut microbial community. Polysaccharide degradation is one of the core functions encoded in the microbiome. Broad expansion of the genes and operons dedicated to degrading and consuming polysaccharides has occurred within the genomes of microbiota-resident species, a logical outcome of the intense competition for these resources. It is, therefore, expected that alterations in the type and quantity of polysaccharides consumed can result in changes in the microbiota community composition and function.

Inulin- and levan-type fructans (homopolymers of β2-1 or (β2-6 fructose units, respectively) are common dietary plant polysaccharides that feed the intestinal microbiota. Multiple bacterial taxa in the gut utilize fructans, including members of Firmicutes, Bacteroides, and Bifidobacterium, and dietary fructan can result in expansion of Actinobacteria, Firmicutes, or Bacteroides. Lack of predictability in how the microbiota responds to such dietary interventions reflects our limited understanding of nutrient sensing and utilization by members of the intestinal microbiota.

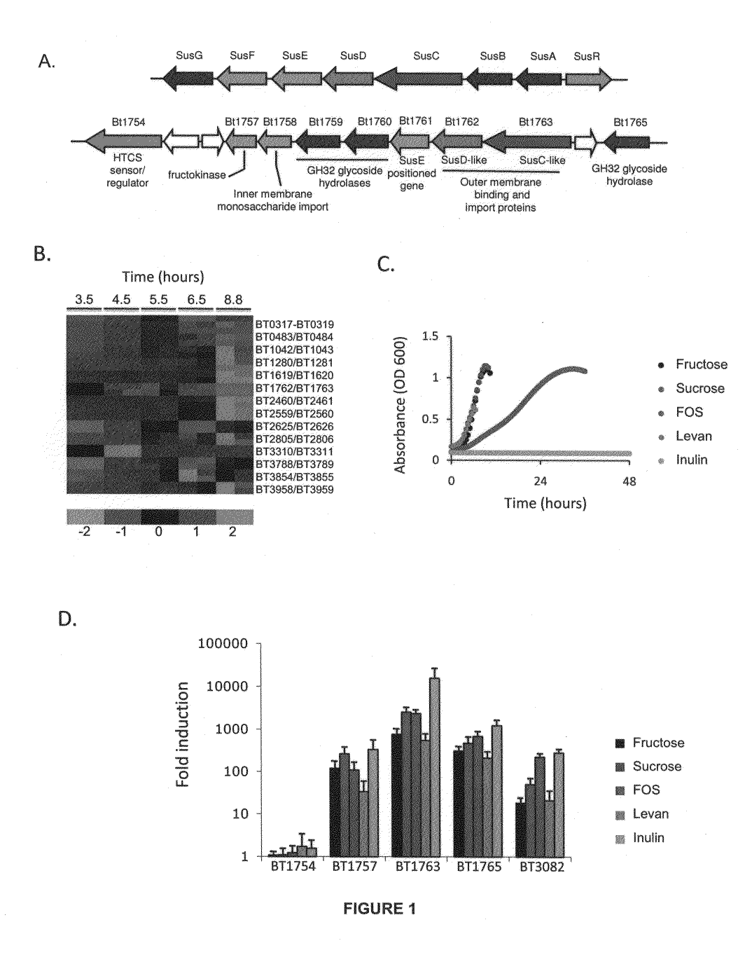

Bacteroides, a major genus in the human microbiota, have a widely expanded capacity to use diverse types of dietary polysaccharides. Much of the glycan degrading and import machinery within Bacteroides genomes are encoded within clusters of coregulated genes known as polysaccharide utilization loci (PULs). B. thetaiotaomicron (Bt), a prototypic member of the Bacteroides, possesses 88 PULs, which differ in polysaccharide specificity. The defining characteristic of a PUL is the presence of a pair of genes homologous to Bt susD and susC, which encode outer membrane proteins that bind and import starch oligosaccharides, respectively (FIG. 1A). The pair of susC and susD homologs is usually associated with genes that encode the machinery necessary to convert extracellular polysaccharides into intracellular monosaccharides, such as glycoside hydrolases (susA, susB, and susG in FIG. 1A). In addition to machinery for polysaccharide acquisition, most PULs contain, or are closely linked, to a gene or genes encoding an inner membrane associated sensor-regulator system, including the novel hybrid two-component systems (HTCS). Bt's genome encodes 32 of these HTCS, which may mediate the rapid and specific responses required in the dynamic nutrient environment of the intestine.

Here we dissect a Bt PUL required for utilization of fructans to better understand how Bacteroides species acquire and process this common class of dietary carbohydrates. In addition, we provide evidence that the associated HTCS controls the expression of the fructan PUL and that monomeric fructose is the activating signal that binds directly to the periplasmic sensor domain of the regulatory protein. These data provide the first example of a well-defined ligand for a member of this class of novel sensor regulators. The fructan PUL is conserved to varying extents among Bacteroides species, corresponding to a range of fructan utilization capability across the genus. Using model intestinal microbiotas living within gnotobiotic mice, we demonstrate that dietary fructan can have disparate effects on community composition, depending upon the fructan degrading capacity of members of the microbiota. These studies demonstrate that within personal microbiomic datasets, genetic biomarkers of discrete functions can be identified. Inference of function from these biomarkers will provide predictive power in determining how an individual's microbiota will respond to changes in diet and other interventions.

### Results

BT1757-BT1763 and BT1765 form a putative polysaccharide utilization locus (PUL) that is transcribed early in Bt's growth in rich media. BT1757-BT1763 and BT1765 encodes eight open reading frames on the negative strand of the Bt genome, including one susC/susD homolog pair (BT1763 and BT1762), a putative outer membrane lipoprotein (BT1761), a putative inner membrane monosaccharide importer (BT1758), a putative fructokinase (BT1757), and three putative glycoside hydrolases (BT1759, BT1760, BT1765) (FIG. 1A). These glycoside hydrolases are members of the Glycoside Hydrolase Family 32 (GH32), a family of enzymes specific for fructans. One of these, BT1760, possesses a N-terminal lipidation motif and is predicted to reside on the cell surface; the other two, BT1759 and BT1765, are predicted to be periplasmic and intracellular, respectively. Directly adjacent to the locus is a putative inner membrane-associated sensor regulator of the HTCS family, BT1754. These data suggest that this PUL encodes the proteins required for Bt's use of fructans. Expression profiling of Bt in rich medium has revealed the upregulation of several PULs, each of which is confined to a discrete phase of growth.

Analysis of Bt transcriptional profiles at five time points that spanned from early log to stationary phase in vitro in rich medium, compared to basal expression in minimal medium containing glucose as the sole carbohydrate (MM-G), revealed that 14 pairs of susC/susD homologs were induced greater than 20-fold at one or more time points during the growth (FIG. 1B). The putative fructan PUL showed upregulation early in Bt's growth suggesting it is responsive to a high priority substrate accessed early in growth on rich medium (FIG. 1B). Genes within this PUL are coexpressed both in vitro in rich medium and in vivo in Bt mono-associated gnotobiotic mice fed a polysaccharide-rich diet, consistent with the functional relatedness of adjacent genes and operon predictions in Bt. Bt increases expression of this PUL in vivo while downregulating the vast majority of other PULs when bi-associated in the gnotobiotic mouse intestine with the methanogenic archeon, Methanobrevibacter smithii. The upregulation of the putative fructan PUL is concomitant with increased densities of Bt in vivo, suggesting that expression of this locus is associated with growth potentiation of Bt.

Bt upregulates its putative fructan PUL when grown on fructose-containing carbohydrates We inoculated minimal medium containing specific fructose-based carbohydrates as the only carbon and energy source with Bt to test if the bacterium is competent to grow on fructans. Bt grew on a broad range of fructose-based glycans, including free fructose, sucrose, levan (high MW fructose polymer with predominantly β2-6-linkages), and fructo-oligosaccharides (FOS; short-chain β2-1 polymers of 2-10 fructose units) (FIG. 1C). However, Bt grew poorly on inulin (β2-1 fructose polymer with an average degree of polymerization of ˜25), with growth only apparent three days after inoculation. Doubling times on simple monosaccharides and disaccharide were similar to one another. In contrast, growth rates of Bt between the different fructans showed large linkage-dependent differences: β2-6 levan resulted in the fastest doubling time (2.7 h), while β2-1 FOS and inulin were significantly slower (doubling times of 5.6 h+/−0.004 and 96.4 h+/−0.05, respectively).

To determine whether these fructose-based substrates induced expression of genes associated with the putative fructan PUL, Bt was grown in either glucose or one of five fructose-containing substrates (fructose, sucrose, levan, FOS, or inulin) as the sole carbohydrate. Cells were harvested at mid-log phase for quantitative RT-PCR (qPCR) analysis, and RNA levels of the 3′ and the 5′ ends of the operon, BT1757 (encoding the fructokinase) and BT1763 (encoding the SusC-like protein), respectively, were used as an indicator of PUL expression (FIG. 1D). Both BT1757 and BT1763 were dramatically up regulated in all media containing fructose, whether as a free monosaccharide or in glycosidic linkage. Across all conditions, expression of BT1757, BT1763 and BT1765 showed coordinated increases consistent with the predicted operon structure. However, BT1754 (the PUL associated putative HTCS) showed no significant induction under all conditions tested. Therefore, the operon that encodes the structural genes of Bt's putative fructan PUL is transcriptionally responsive to fructose-containing carbohydrates. Published surveys of Bt gene expression in numerous carbohydrates support that up regulation of the fructan PUL is specific to fructose-containing substrates. Two genes within Bt's genome that are not physically associated with the putative fructan PUL, a second putative periplasmic GH32 (BT3082) and a second putative fructokinase (BT3305) were likely candidates to be involved in fructan utilization.

Analysis of BT3082 and BT3305 expression by qPCR revealed that BT3082 was induced in all fructose-containing media and showed a pattern of induction consistent with those seen for BT1757, BT1763, and BT1765 (FIG. 1D); however, BT3305 showed no change in expression or a slightly reduced expression in all conditions. These data suggest that the fructosidase, BT3082, but not the putative fructokinase, BT3305, is part of the regulon of the putative fructan PUL.

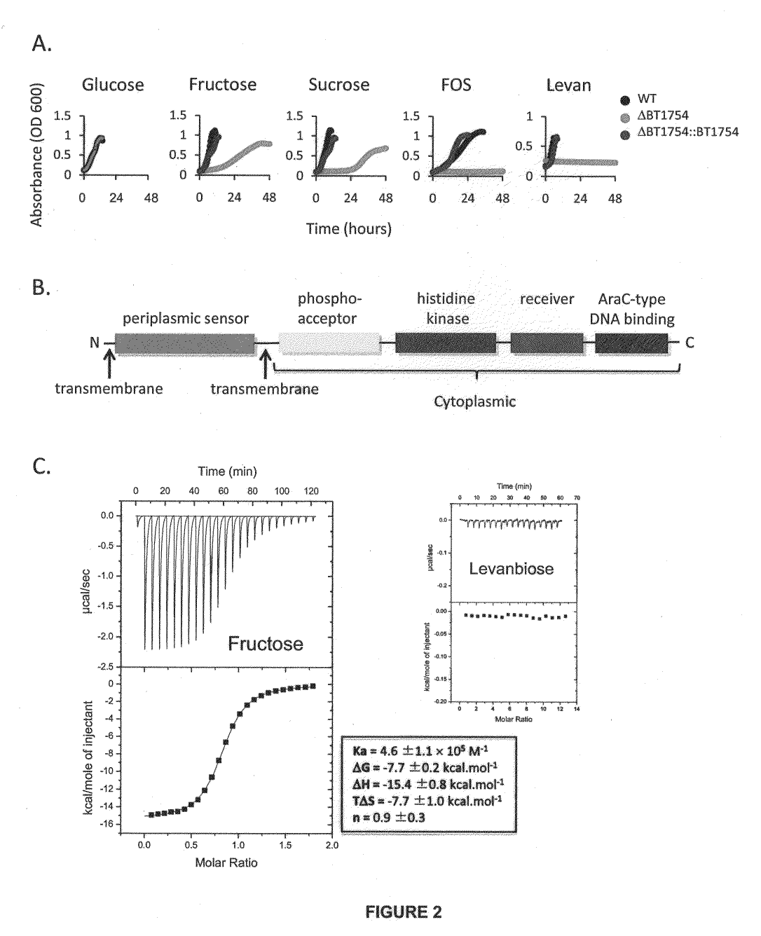

The hybrid two-component system BT1754 is required for efficient fructan utilization by Bt. We assessed the ability of an isogenic mutant of Bt lacking the BT1754 gene to grow in a panel of fructose-based minimal media (MM) to test if upregulation of the PUL was dependent upon the HTCS signaling sensor. An in-frame, unmarked deletion of BT1754 was constructed using a standard counter-selectable allele-exchange procedure. Bt-ΔBT1754 exhibited normal colony morphology on solid medium and grew with a similar doubling time to wild type in MM-glucose (2.6 h); however, Bt-ΔBT1754 failed to grow in any of the three fructans (FOS, inulin and levan) and showed retarded growth in fructose and sucrose (FIG. 2A). Additionally, Bt-ΔBT1754 does not exhibit prioritized up regulation of the putative fructan PUL during growth in rich media. Complementation of this mutant was achieved by introducing the genomic fragment containing BT1754 and its 5′ intergenic upstream promoter region in trans. Growth of the ΔBT1754::BT1754 complemented mutant restored growth in all fructose-based media to levels comparable to wild type (FIG. 2A). These data demonstrate the HTCS encoded by BT1754 is required for Bt's use of fructans.

The periplasmic domain of the hybrid two-component system BT1754 binds to monomeric fructose. One of the key unanswered questions concerning the HTCS family, and many extracellular sensory systems, is the identity of the molecular triggers for signaling events. The predicted innermembrane localization of Bt's HTCS family members, including BT1754, suggests that the periplasmic region likely serves as the sensor/receptor, similar to classic two-component systems. Analysis of the sequence of BT1754 revealed a typical HTCS architecture with an N-terminal predicted periplasmic sensor domain flanked by two transmembrane regions and a C-terminal cytoplasmic histidine kinase domain, a phosphoacceptor domain and a response regulator (including a receiver and an HTH_AraCtype DNA binding domain). (FIG. 2B). Uniquely within Bt's HTCS, the sensor domain displays homology to Type I bacterial periplasmic binding proteins (PBPs). As PBPs are known to bind small molecules such as sugars, we expressed the periplasmic domain of BT1754 (BT1754-PD; residues 29-343) in a recombinant form and tested for binding to a range of monosaccharides and fructan-derived oligosaccharides to see if direct interaction with a specific carbohydrate is the means of signal perception in BT1754. The isothermal calorimetry data reveal that BT1754-PD binds specifically to fructose, with a Kd of ˜2 μM and a stoichiometry of 1:1 and does not interact with either β2-1- or β2-6-linked fructooligosaccharides or any other monosaccharides, including glucose and ribose (FIG. 2C).

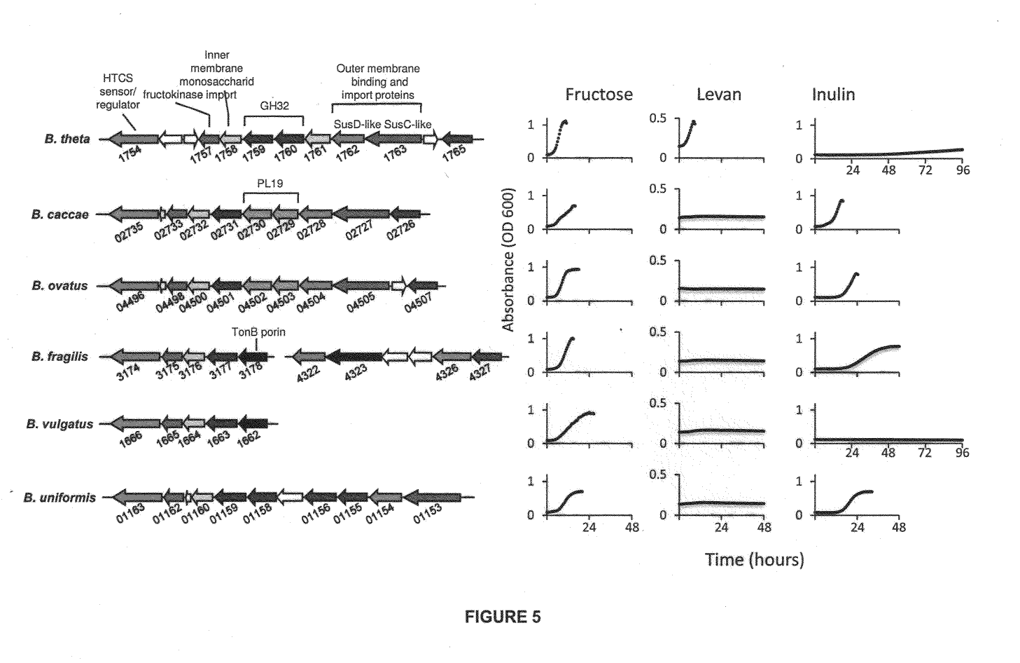

The fructan PUL is variably conserved in sequenced Bacteroides, which have differing capacity to utilize fructan We performed a comparative genomic analysis focused on Bt's fructan utilization locus between five sequenced species of Bacteroides to gain further insight into the mechanism of fructan use for this major group of gut resident microbes. Using the N-terminal fructose-binding domain of the HTCS BT1754 to query a BLAST database consisting of the Bacteroides species B. caccae, B. vulgatus, B. uniformis, B. fragilis, and B. ovatus, we have identified a single orthologous HTCS in each species, with the exception of B. fragilis, which harbors two BT1754-like genes. Sequence identity between the periplasmic sensor domains of the BT1754 orthologs was high for all but one, ranging from 93% for the B. ovatus protein to 58% for the B. vulgatus domain. Furthermore, the residues involved in fructose binding in BT1754 are almost completely conserved among orthologs, consistent with conservation of the ligand sensed by each HTCS. The periplasmic domain of one of the two B. fragilis orthologs (BF4326) displayed only 36% identity with BT1754-PD, and this domain was unique in its lack of fully conserved fructose binding residues. Regions adjacent to the HTCS in each genome were analyzed and found to display local synteny with the Bt locus (FIG. 5, left panel), including the presence of open reading frames that are predicted to play a role in utilization of fructosecontaining carbohydrates.

In all six Bacteroides species, the HTCS is adjacent to a predicted fructokinase, a putative inner membrane monosaccharide importer, and GH32-family glycoside hydrolases. In each genome, except that of B. vulgatus, the syntenic regions also contain a susC/susD homologous pair. The presence of an apparent fructan PUL in multiple Bacteroides species suggested that fructan utilization is shared between members of this genus. Testing for growth on fructose-based glycans revealed that all six species are competent for growth on fructose (FIG. 5, right panel), sucrose and FOS. All Bacteroides species tested, except B. vulgatus, were able to grow efficiently using one of the long-chain fructans, inulin or levan. The inability of B. vulgatus to grow on long-chain fructans is consistent with the absence of a susC/susD-like pair within its locus. B. caccae, B. ovatus, B. fragilis and B. uniformis can utilize inulin with efficiency similar to their use of glucose. This contrasts with Bt inulin use, which is only observed after three days (FIG. 5). Bt was the only species tested able to use levan, which was particularly striking when considering the overall similarity in PUL structure between Bt, B. caccae, and B. ovatus. However, examination of PUL gene content of the two inulin-utilizing species revealed genes encoding PL19 enzymes, a family, that is known to include members capable of degrading the β2-1 fructan. Additionally, Bt's extracellular β2-6-specific GH32, BT1760, does not possess an orthologous gene in the other species. Notably, two other sequenced Bt strains utilize levan more efficiently than inulin in vitro, similar to the type strain. Both of these strains possess orthologs to the type strain's BT1760. Together these data demonstrate that differences in fructan specificity of Bacteroides species correspond to differences in the gene content of their respective fructan PULs.

Genomic content of Bacteroides species predicts changes in microbiota composition induced by an inulin-based diet. The differences in ability to utilize fructans between the Bacteroides species implies that the relative success of a species within a gut ecosystem may be determined, in part, by the abundance and type of fructan in the host diet. Furthermore, the comparison of genomic sequences and differences in fructan use between species suggests that personalized predictions of microbiota response to specific dietary polysaccharides may be made based on metagenomic microbiome sequence data. We constructed defined two-member communities of Bacteroides species within the intestines of gnotobiotic mice to test how model microbiotas respond in vivo to dietary inulin, which, unlike levan, is available in pure form in quantities sufficient to conduct such a study. Our in vivo experiment aimed to test how differing functionalities embedded within the genomes of two different two-species model microbiotas influence inulin-induced changes in community composition. Due to B. caccae's superior ability to use inulin compared to Bt, we tested whether B. caccae would become dominant over Bt within the intestines of mice fed an inulin-supplemented diet. Conversely, Bt's poor growth on inulin is better than B. vulgatus, which is unable to utilize inulin, suggesting that Bt might benefit from inulin when colonized with B. vulgatus.

Two groups of 8-12-week old, germ-free mice were colonized with equivalent quantities (10⁸ colony forming units, CFU) of Bt and B. caccae or Bt and B. vulgatus. Each mouse was maintained on a standard polysaccharide-rich diet for the first 7 days of colonization and then switched to a diet in which the sole polysaccharide was inulin (10% w/w) for an additional 14 days. Mice were individually housed throughout the experiment to ensure no cross inoculation could occur and bedding was changed every two days. Total bacterial colonization density was determined by assessing the CFUs in feces over 21 days. The change in each species' relative abundance before and after dietary inulin supplementation was assessed using species-specific primers in a quantitative PCR assay.

Our results disclosed that total fecal bacterial densities over the course of the experiment did not differ significantly upon dietary shift (total densities ranged from 10¹⁰-10¹¹ bacteria/ml of fecal material). Relative densities were determined on days 4 and 6 (standard diet) and on days 13 and 21 (6 and 14 days after dietary switch). In the Bt/B. caccae, bi-associated mice, before the diet switch (day 6 post-colonization in mice fed a standard diet), Bt comprised 87±3% of the community, indicating that Bt is better adapted than B. caccae to these in vivo conditions. Six days after a change to the inulin-based diet, Bt levels dropped to 80±4%, and B. caccae increased to 20±4%. After two weeks consuming the inulin diet, the relative proportion of the two species showed a more drastic shift in favor of B. caccae: Bt representation decreased to approximately 49±6% vs. 51±6% B. caccae (p=8×10⁻⁵, day 21 versus day 6; n=7 mice; Student's t-test).

In contrast, the Bt/B. vulgatus bi-associated mice did not exhibit any significant trend in changed community composition after 6 days on an inulin-based diet, but Bt increased in abundance from 74±3% on day 6 to 84±5% on day 21 (p=0.1; n=3 mice) on the inulin enriched diet. The delayed and modest effect of diet influencing the composition of the Bt/B. vulgatus bi-association is consistent with poor inulin use by Bt and no inulin use by B. vulgatus. Together these data are consistent with dietary polysaccharide-induced changes in the microbiota composition that are predictable based on the resident species' ability to use that polysaccharide.

In the previous experiment, inulin was the sole polysaccharide in the diet. We wondered whether we would observe the same inulin-induced increase in B. caccae relative to Bt if other polysaccharides were also present in the diet. To test this, gnotobiotic mice were co-colonized with Bt and B. caccae and maintained on the standard diet with inulin supplementation in the water (1% w/v). Over the 14 days the mice ingested an average of 117±6 mg of inulin daily via the water (compared to 355±7 mg/day with the inulin diet). Fecal samples were tested by qPCR over the course of the 21-day experiment for relative levels of Bt or B. caccae. These data revealed no statistical difference in the change in relative colonization between mice fed inulin-supplemented water compared to controls that received the same standard diet for 21 days, but received no inulin. These data suggest that when mice were fed a diet rich in carbohydrates, the presence of inulin did not provide enough of an advantage to B. caccae to allow it to out-compete Bt; however, the amount of inulin supplied in the water (117 mg/day average) was less than the amount derived from the inulin diet (355 mg/day average) potentially contributing to the lack of the B. caccae response.

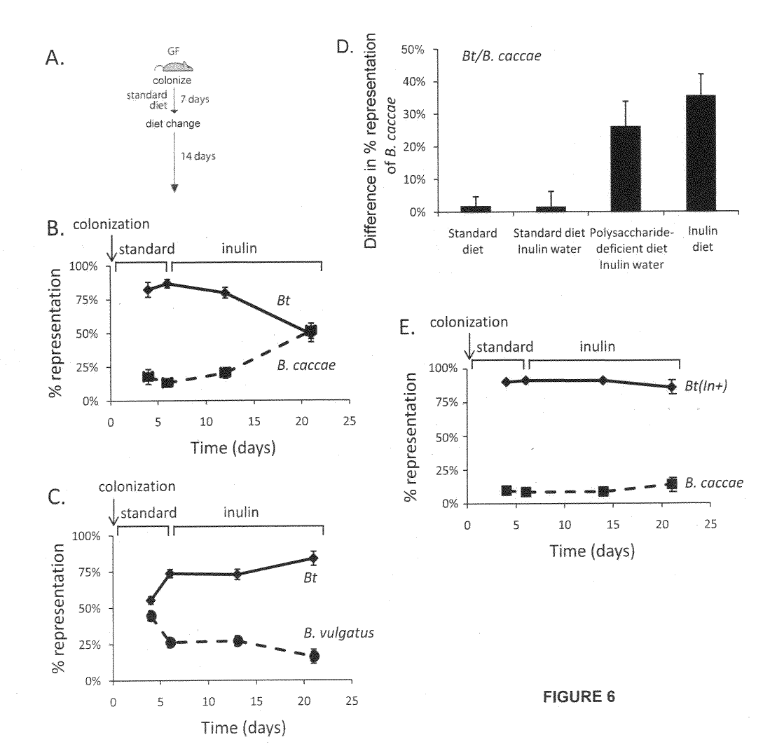

We decided to feed mice a custom diet deficient in all polysaccharides and supplement inulin in the water to determine whether a lower dose of inulin in the absence of other polysaccharides was sufficient to provide B. caccae a competitive advantage over Bt in vivo. Under this experimental paradigm the mice consumed an average of 97 mg of inulin per day. After. 14 days on inulin-water supplementation, the proportion of B. caccae increased by 26±8% (FIG. 6D). While not as robust an increase as observed in the inulin-only diet experiment (which showed a 36±7% increase in B. caccae), these data demonstrate that reduced inulin consumption in the absence of competing polysaccharides, offers a significant competitive advantage to inulin-utilizing B. caccae, consistent with the flexible nutrient foraging the Bacteroides species exhibit. The wide range of polysaccharides present in the standard diet allows Bt to compete effectively with B. caccae even in the presence of inulin.

We finally demonstrate the importance of inulin utilization for conferring a competitive advantage in hosts fed an inulin-rich diet using a genetic proof of this effect. The region of the B. caccae fructan utilization locus from the susC-like gene through the GH32-encoding gene (BC02727-BC02731) was cloned and expressed in a strain of Bt that is compromised in its ability to utilize levan (Bt-ΔBT1763) under the control of the BT1763 promoter. The resulting strain, Bt(In+), exhibits efficient growth in minimal medium containing inulin, similar to B. caccae. Repeating our original in vivo competition experiment with Bt(In+) revealed that conferring inulin use ability upon Bt eliminates the ability of B. caccae to become dominant in the presence of an inulin-based diet (FIG. 6E). This result confirms that the specificity of dietary polysaccharide use is the key functionality that dictates the alterations in the model microbiota that we observe.

These results support that changes in microbiota community membership brought on by dietary change can be inferred based on genomic and functional knowledge of resident microbial populations. They also demonstrate that diet can be a dominant determinant in dictating changes in microbiota composition.

Inulin (β2-1 fructan) and levan (β2-6 fructan) are polysaccharides that are abundant in the human diet, but are resistant to host-mediated digestion in the upper gastrointestinal tract. These glycans instead serve as a carbon and energy source for the bacteria that reside in the distal intestine. Bacteroides thetaiotaomicron, a resident of the human GI tract, encodes a fructan utilization locus, BT1757-63 and BT1765, the gene products of which enable efficient acquisition and use of levan-type carbohydrates.

The fructan PUL is adjacent to a hybrid two-component system sensor-regulator, BT1754, which binds only to monomeric fructose, a signal sufficient to induce transcription of the locus. Bt appears to use the liberated fructose as a proxy (i.e., indicator) for fructan, which results in upregulation of the machinery to utilize the polysaccharide. This is consistent with previous data that demonstrate Bt's constitutive, low-level expression of signaling sensors and glycoside hydrolases in conditions lacking the relevant substrates, as well as the low level cell surface levanase activity we observe with whole cells grown in glucose. The constitutive expression suggests that Bt employs a strategy of being prepared to degrade multiple polysaccharides immediately upon their arrival into the distal gut environment.

Specific liberated carbohydrates that result from the degradation serve as signals that augment expression of the appropriate PUL via a specific sensorregulator such as a HTCS. The binding of BT1754 to monomeric fructose also results in a failure of the sensor to differentiate β2-1 and β2-6 linkages despite Bt being much more efficient in use of the levan-type fructans. Specificity of signal is instead derived from the cell surface structural components of the PUL, which serve as the “gateway” for substrates crossing the outer membrane. The cell surface SusD homologue, BT1762, the susE-positioned gene product, BT1761, and the endo-levanase, BT1760, all contribute to the specific import of β2-6 fructans into Bt's periplasm. BT1754 relies upon the specificity of the cell surface polysaccharide degradation and binding machinery to provide fructose derived from β2-6 fructan to the periplasm where the sensor is sequestered. Despite Bt's inability to utilize inulin efficiently it is able to grow well on FOS, a short chain β2-1 fructan. Notably, the fructan PUL of Bt is up regulated during growth in vitro in minimal medium containing FOS or inulin. Bt's ability to grow in FOS at a rate that is significantly faster than inulin is likely due to the difference in degree of polymerization between the two substrates.

Among the Bacteroides species tested, Bt appears to be unique in its ability to utilize levan, whereas other species are adept at utilizing polymeric β2-1 fructans. Such phenotypic differences, combined with dietary variation between individuals, could provide the basis for the striking person-to-person variability observed for Bacteroidetes in human microbiota enumeration studies. Our in vivo studies using fructan-enriched diets illustrate that species well-adapted to use inulin gain a competitive advantage when hosts are fed an inulin-based diet. These results suggest that some aspects of diet-induced changes in microbiota composition may be predetermined based on the intrinsic capacity of an individual species to use the substrates that are being consumed by the host.

As the age of personal genomes approaches, some aspects of diet and medical therapies will be customized based on genotype. Diet can also be personalized to optimize microbiota function and interaction with the host based on the metagenomic analysis of an individual's microbiota. A prerequisite for incorporating vast amounts of microbial genomic data into personalized, preventative medicine is to attain a mechanistic understanding of the most dominant aspects of microbiota function. Here we present a case study of how understanding the mechanisms that link the microbome to microbiota function can enable individualized predictions of microbiota response to perturbations.

We have taken two-species model microbiotas that collectively possess close to 10,000 genes and predicted how they will respond to a specific dietary cue based on a functional understanding of the ˜20 relevant genes. A similar distillation of full microbiomic datasets that contain >10⁶ genes, to a relevant subset, will be required to make microbiota management tractable. With an ever-increasing understanding of how the biology of host and microbiota integrate, we may able to use genomic and microbiomic sequence data to intentionally program or re-program the emergent properties of the host-microbial superorganism.

### Materials and Methods

Culturing bacteria. Bacteria were cultured in TYG and MM as described previously. The following bacteria were used: Bt (VPI-5482), B. caccae (ATCC-43185), B. ovatus (ATCC-8483), B. fragilis (NCTC-9343), B. uniformis (ATCC-8492), and B. vulgatus (ATCC-8482). Growth curves in MM were obtained using a Powerwave (Biotek) reading OD600 every 30 min from anaerobic cultures at 37° C.

Quantitative RT-PCR analysis. Quantitative RT-PCR was performed using gene-specific primers as described previously with SYBR Green (ABgene) in a MX3000P thermocycler (Strategene). Gene deletion and complementation in Bt In-frame (non-polar) gene deletions for mutants were generated using counter-selectable allele exchange. PCR amplified genes for complementation were ligated into the pNBU2-tetQb vector and conjugated into Bt via E. coli S17.1λ-pir. Resulting clones were screened by PCR and sequenced to confirm isolates.

Gene cloning. Genes for expression were amplified from Bt genomic DNA using the primers stated in Table S3 and cloned into pRSETA (Invitrogen) or pET22b (Novagen).

Protein expression and purification. Recombinant proteins were expressed in E. coli C41 or BL21 cells and purified in a single step using metal affinity chromatography as described previously.

Sources and preparation of carbohydrates. Monosaccharides, sucrose, and chicory inulin for enzymatic and binding assays were obtained from Sigma. Growth of Bacteroides strains, qRT-PCR; and mouse experiments used inulin, FOS (Orafti-Beneo group; OraftiHP, OraftiP95, respectively) and levan (Sigma; 66674). Kestooligosaccharides were from Megazyme. Levanoligosaccharides were produced by partial acid hydrolysis (1M HCl at 25° C. for 20 min-1 h) of levan (Montana Polysaccharides). NaOH-neutralized samples were separated on BioGel P2 (BioRad) size exclusion resin.

Isothermal titration calorimetry. Measurements were carried out essentially as described previously, except that a Microcal VP-ITC machine was used, and proteins were dialyzed into 20 mM Tris-HCl, pH8.0. The assumption that n=1 for BT1762 binding to levan was based on the structure of the starch binding SusD.

Thin layer chromatography. Samples were spotted onto foil backed silica plates and placed in a glass tank equilibrated with butanol:acetic acid:H₂O (2:2:1). Sugars were visualized using orcinol-sulphuric acid (sulphuric acid:ethanol:water 3:70:20 v/v, orcinol 1% w/v), 90° C. for 5-10 min.

Enzyme assays. All assays were carried out at 37° C. in 20 mM Tris-HCl, pH8.0. Activity of BT1760 was determined by quantifying the amount of reducing sugar released using the DNSA assay. Free fructose was determined using a modified fructose detection kit (Megazyme International). Kinetic parameters were determined by fitting initial rates vs. substrate concentration (measured at six substrate concentrations that spanned the KM) to a non-linear model of the Michaelis-Menten equation (Graphpad Prism, v5.0).

Enzyme localization studies. Cultures grown on 0.5% (w/v) fructose or glucose were harvested by centrifugation (OD₆₀₀˜1.0). PBS washed cells and 0.5% levan or inulin in 20 mM Tris-HCl, pH8.0, were incubated at 37° C. Reducing sugar present was quantified using DNSA reagent. Activities of the periplasmic marker alkaline phosphatase and cytoplasmic marker glucose-6-phophate dehydrogenase were compared to lysed cells to ensure no cell lysis/leakage occurred.

Bacterial colonization and density determination of germ-free mice. Germ-free Swiss-Webster mice were maintained in gnotobiotic isolators and fed an autoclaved standard diet (Purina LabDiet 5K67) or custom diet (Bio-Serv, http://bio-serv.com/), in accordance with A-PLAC, the Stanford IACUC. Mice were bi-associated using oral gavage (10⁸ CFU of each bacterial species). Relative densities of bacteria were determined by qPCR using strain-specific primers.

### Example 2

### Discovery of Gut Microbiota-Targeted Small Molecules

### New Tools and Therapeutics

While the gut microbiota achieves a density greater than other natural microbial ecosystems, such as soil, it is surprisingly less diverse than one might suspect. Fewer than 15 of the 70 or more known bacterial divisions are known to be represented in the human microbiota, and two of these, the Bacteroidetes and Firmicutes, constitute more than 90% of the total community. The microbiota composition varies with host genotype, diet, age, health status, and a number of other variables. Altered microbial composition has been linked to inflammatory bowel diseases and obesity in mouse models and in humans, and oral antibiotic use results in long-term disruption of the microbiota. While such changes in our microbial composition may not be desirable, these studies clearly illustrate that our microbiota is in a dynamic state and susceptible to manipulation. This plasticity may be harnessed for rational manipulation to benefit host health.

Much of the current effort in understanding the human microbiota is focused on sequence-based characterization. Several enumeration studies have concentrated on defining “normal” versus “disease associated” microbiota composition using ribosomal RNA sequencing, which provides culture-independent molecular signatures of the constituent species. Additionally, large-scale efforts are underway, such as the Human Microbiome Project, to identify and catalog the genome sequences of microbial species associated with the human body. These studies promise to make important strides in defining the commensal microbial species associated with health and disease.

Despite the rapidly accumulating genomic and enumeration data, distinct gaps in our understanding of the gut microbiota that are not addressed by these ongoing efforts will persist. The development of new tools that permit the controlled manipulation of the microbiota will be required to address such fundamental questions.

The present invention addresses how perturbations in the intestinal environment, such as changes in host diet, community composition, and host genotype, alter microbiota structure and function, and how these changes, in turn, influence host biology.

In part these studies will rely upon a gnotobiotic mouse models, where specific compositions of microbiota are known. A particularly useful and relevant model is a ‘humanized’ gnotobiotic mouse, consisting of ex-germ-free mice that have been colonized with microbes from the human microbiota or representing a profile approximating the features of a human microbiome. Germ-free mice offer an ideal platform to create model microbial communities that are relevant to the human condition. Parameters of the host peripheral and mucosal responses are defined in ex-germ-free mice that are colonized with (i) discrete subsets of the species that constitute the human microbiota, which provides one way to mimic aberrant overgrowth of particular species and allows for functional genomic characterization of microbial community members, and (ii) a complete human microbiota, followed by perturbations caused by dietary shift, pathogen exposure, antibiotic treatment, and microbiota-targeted small molecules.

Small molecules or other compounds or components that could be used as drugs or supplements are identified using high-throughput cell based screening of anaerobic bacteria that that are indigenous to the human anatomical areas. Identification of such compounds provides lead compounds that can be developed for a new class of therapeutics: microbiota-targeted drugs or supplements. Additionally, this approach enables basic studies in which the microbiota can be rationally manipulated.

In some cases it may be desirable to inhibit species-specifically. In other cases it may be particularly useful to target broadly, for example by inhibiting nutrient utilization pathways common to various species.

### Example 3

### General Overview of Screening Strategy

In the screening strategy and concept of the invention, cells of one or a defined set of microbial species are exposed to a candidate agent within an anaerobic environment. In one embodiment, the screen is conducted in a high-throughput format such as within a 384-well plate, where a panel of candidate agents may be tested; with one or a defined set of candidate agents within each well. Alternatively, screens could be conducted on a semi-solid surface, such as an agar-based medium. Cell-based screens are designed to allow the identification of candidate agents that impact the microbe(s) in at least one of numerous ways. In some instances the desired impact is alteration in microbial growth or metabolism, either inhibition or potentiation. In some cases, candidate agents that inhibit one aspect of metabolism but potentiate another aspect (e.g., redirection of metabolic flux) are of interest. Agents may also be screened for the ability to alter other aspects of cell physiology, including but not confined to cell permeability, cell shape, motility, or attachment to a substrate, coated surface, or other cells. Screens may be designed to identify agents that alter the activity of specific signaling pathways (e.g., agonists or antagonists) or that elicit or inhibit a desired set of microbial responses through other direct or indirect means, such as the production of a specific small molecule, protein, carbohydrate, or lipid. In all cases, the screen will be designed to permit automated or high-throughput detection of the response. In the simplest form, this will be an alteration in optical density (change in cell growth, size, or shape). In other embodiments, the readout could be colorimetric detection of an enzymatic activity, or inhibition thereof, expression of a reporter gene, production, conversion, or loss of another entity (metabolite, protein, carbohydrate, lipid, etc.) that is detectable through another method (e.g., ELISA; mass-spectrometry, exposure of microbial culture to another cell-based reporter system, etc.)

In an additional embodiment, a secondary screen, and multiple additional screens, are conducted on other microbial species or consortia that share characteristics with the species or community of interest. Additional species may be close phylogenetic relatives (e.g., another member of the genus) or may be more distantly related but share genomic features or general aspects of microbial cell biology or physiology with the primary species of interest. In the case of defined consortia, the identity of species may or may not be similar between primary and additional screens, but the communities will share some collective feature or features of interest. The goal of performing similar screens on additional microbes is to identify agents that are able to exert an impact more broadly than on one individual species. By design, this latter strategy will select for compounds that act across various species. Without being bound by a theory, the mostly likely mechanism for such activity would be binding to highly conserved regions of proteins and is more likely to operate effectively on closely related species and in proteins of conserved function. The prevalence of lateral gene transfer events in dense microbial communities suggests that agents may also act across multiple distantly related species that share genomic features. In cases, convergence of function may also render to unrelated targets susceptibility to the same agent or agents. Such mechanisms are likely to be less susceptible to resistance development by mutation, and thus a specific embodiment of the invention relates to a method of identifying microbe-targeted drugs with a reduced resistance profile. Adaptation of standard high-throughput screening methods for compatibility with anaerobic bacteria utilizes an integration of technology and instrumentation as described below.

In one example, small molecules are arrayed into 384-well plates containing defined medium plus one of two specific carbon sources, an experimental carbon source or a control carbon source. Plates are transported into a Coy anaerobic chamber that contains a liquid handler, plate reader, and 37° C. incubator and allowed to pre-equilibrate overnight (16 h) within humidified chambers. Cultures of two related microbial species (e.g., two Bacteroides species, see below) are grown from frozen aliquots for a predetermined period of time in a controlled environment. After culture growth and plate equilibration, these cultures are inoculated into each well, using an automated liquid dispenser (e.g., Multidrop). The screen is conducted at 37° C. within humidified chambers in an anaerobic chamber, and growth measured (optical density at 600 nm) at appropriate intervals. Growth inhibitors are identified that are specific for the experimental but not the control carbon source, and are active across both species. These candidates are re-screened using identical methodology at eight different concentrations to establish a dose-response curve.

### Example 4

### Inhibitor Screening

One commonly attempted strategy for altering the composition of the human microbiota is through the ingestion of prebiotic compounds. Prebiotics, which are typically plant-derived polysaccharides, are ingested for the purpose of stimulating growth of specific taxa within the microbiota. Plant polysaccharides derived from host diet that are resistant to host digestion (e.g. dietary fiber) reach the distal gut where they are a coveted energy source for commensal microbes. Changes in host consumption of different classes of polysaccharides can result in changes in microbiota community composition as a result of preferential expansion of microbial components that are well-adapted to grow on polysaccharides that are abundant in host diet. However, attempts to manipulate the microbiota composition using prebiotic polysaccharides have resulted in unpredictable outcomes. Due to their lack of specificity, prebiotics provide poor control over microbiota structure and function.

One strategy for attaining improved specificity of prebiotics is to identify small molecules that chemically ablate the machinery required for utilization of the specific prebiotic, for taxa that are exhibiting unwanted expansion. This inhibition of a nutrient utilization pathway within a specific taxon imposes a competitive disadvantage upon it and allows preferential expansion of other desired taxa. With an increasingly detailed understanding of the mechanisms employed by prominent members of the microbiota for nutrient sensing and acquisition, identification of such small molecules is now tractable. These types of specific mechanistic insights have given rise to the screening concept of this invention. However, the generalizeable screening platform of the invention is useful for any microbe in that it does not require mechanistic information for the targeted microbe of interest. On the contrary, the screen is constructed so that any microbe, no matter how little mechanistic information is known about it, can serve as a target against which reliable inhibitor hits can be identified.

Small molecules as selective and reproducible modulators of the microbiota. Bacteroides thetaiotaomicron (B. theta) is an abundant member of the human microbiota constituting 6% of >11,000 rRNA sequences obtained in a comprehensive enumeration. Recent genetic studies in B. theta confirm that the many proteins involved in the multi-step process of harvesting, degrading, and metabolizing specific polysaccharide substrates provide a number of therapeutic targets.

One B. theta locus, which is dedicated to the utilization of a common class of fructose-based prebiotics, (fructans), is used in this patent as a representative model system to demonstrate the utility of the current approach by targeting nutrient utilization systems. B. theta's genome sequence revealed one of the largest glycobiomes (genes involved in the degradation, import and metabolism of glycans) of any sequenced bacteria, including 241 glycoside hydrolases, the enzymes that degrade polysaccharides into bioavailable component monosaccharides. These glycoside hydrolases are localized to polysaccharide utilization loci, which are operons that code for the degradation and import machinery dedicated to specified polysaccharides. The well-described starch utilization system (Sus) is one example of such an operon. SusC and SusD, two outer membrane proteins responsible for starch binding and uptake, are each members of expanded families of paralogous genes. B. theta encodes ˜100 pairs of SusC/SusD paralogs. Typically genes for this binding and import machinery are located immediately adjacent to glycoside hydrolases, genes coding for metabolic enzymes that shunt the liberated monosaccharides into glycolytic pathways, and sensor regulators that serve to perceive and upregulate expression of the cognate locus. Initial analyses of these loci suggest that each is a semi-autonomous unit that encodes all functionality required for a microbe's sensing, acquisition, and catabolism of a distinct polysaccharide substrate.

The SusC/SusD-based polysaccharide utilization loci are ubiquitous in other sequenced species of Bacteroides. In the SusC/SusD based systems, an endo-acting glycoside hydrolase tethered to the outer membrane of B. theta hydrolyzes polysaccharides into small oligosaccharides, which are bound by the SusC/SusD complex. The SusC porin imports oligosaccharides into the periplasm by the SusC porin, where they are processed to mono- and disaccharides by a second glycoside hydrolase. Monosaccharides cross the inner membrane via a specific transporter, and are then converted into glycolytic substrates by the requisite enzymes (isomerases, kinases, etc.)

One of the most commonly used classes of prebiotics, fructans, provides a model for studying the lack of selectivity toward utilization by a specific taxon that many dietary polysaccharides exhibit. These linked linear fructose oligomers are ingested with the aim of increasing the prevalence of members of the Bifidobacterium genus, relative to the more abundant members of Bacteroidetes and Firmicutes. Human dietary fructan supplementation trials have resulted in the expansion of both Bifidobacteria and Bacteroides, which is consistent with both genera containing many species adept at utilizing fructans as the sole carbon and energy source. Furthermore, in vitro and in vivo studies have suggested that fructans are highly coveted by B. theta.

To identify B. theta genes involved in fructan utilization, 4600 random transposon mutants were screened for lack of growth specifically in fructan-based defined medium (i.e. no growth defect in glucose based defined medium): B. theta transposon mutants deficient in fructan utilization were identified in several genes of a novel polysaccharide utilization operon: a hybrid two-component system signaling sensor, a SusC-paralog, and a monosaccharide transporter, confirming that multiple proteins encoded within the locus are viable targets for chemical ablation. In vivo, wild-type B. theta outcompetes an isogenic mutant in the fructan signaling sensor by >40-fold after a 10 day colonization in gnotobiotic mice fed a diet that contains, but is not enriched in fructan. Elevation of fructan in the diet would likely exacerbate the competitive defect of the strain deficient in fructan utilization, therefore this 40-fold growth defect may underestimate the mutant's disadvantage. Together, these results demonstrate that inhibiting a protein encoded within a polysaccharide utilization locus that is involved in harvesting nutrients in vivo will result in attenuated growth and expansion.

Comparison of recently sequenced genomes of multiple Bacteroides species has permitted the identification of orthologous signaling sensor genes and adjacent fructan-utilization operons in multiple members of the genus. Such orthologs were notably absent in other taxa. These results demonstrate that conservation of the widespread ability to utilize fructans among the Bacteroides is the result of a highly conserved operon, unique to the Bacteroides. The fact that many such nutrient utilization operons are shared between Bacteroides species is a key to understanding how a complex microbial community containing hundreds of species can be significantly manipulated via a single small molecule. For example, the conservation of fructan-perceiving and harvesting system of B. theta in other Bacteroides species illustrates that a small molecule targeted to any of the proteins in the pathway could cause a genus wide ablation of the fructan utilization system within Bacteroides. Co-administration of dietary fructan and a Bacteroides fructan utilization inhibitor would permit beneficial Bifidobacterial expansion in the absence of competition from Bacteroides species.

In the screening strategy and concept of the invention, a first screen is conducted to identify compounds that inhibit B. theta growth in defined medium that contains a fructan as the sole carbon source. A simultaneous screen is conducted in glucose containing defined medium to identify the subset of compounds that shows fructan-specific inhibition. While conventional antimicrobial screening for global growth inhibition does not permit isolating inhibitors of pathways of interest, this strategy ensures that the inhibitor is specific to the desired pathway by verifying growth in a counter-screen conducted using a carbon and energy source that is distinct from the prebiotic employed in the screen.

In an additional embodiment, a secondary screen is conducted on the sister species, B. caccae, identical in design to the B. theta screen (including fructan- and glucose-based media) to identify which fructan growth inhibitors are species-specific versus those that inhibit across species. By design, the latter strategy will select for compounds that inhibit across various species. Without being bound by a theory, the mostly likely mechanism for such activity would be binding to highly conserved regions of proteins. Such a mechanism is likely to be less susceptible to resistance development by mutation, and thus a specific embodiment of the invention relates to a method of identifying microbe-targeted drugs with a reduced resistance profile. Adaptation of standard high-throughput screening methods for compatibility with anaerobic bacteria utilizes an integration of technology and instrumentation in order to provide for reproducible screening, as described above, which may utilize media, inocula and incubation conditionsas described herein that provide for the desired reproducible results.

In the current example discussed above, small molecules are arrayed into 384-well plates containing defined medium plus either fructan or glucose. Plates are transported into a 78-inch Coy anaerobic chamber that contains a liquid handler, plate reader, and 37° C. incubator. A saturated culture of B. theta or B. caccae is inoculated into each well, using an automated liquid dispenser (e.g. Multidrop). The screen is conducted at 37° C. in an anaerobic chamber, and growth measured (optical density at 600 nm) at appropriate intervals. Fructan-specific growth inhibitors are re-screened at eight different concentrations to establish a dose-response curve.

Compounds that inhibit both B. theta and B. caccae growth specifically on fructan, as defined above, are tested within gnotobiotic mice harboring model microbial communities composed of members of Bifidobacteria and Bacteroides. The small molecule is given orally to mice in conjunction with a custom mouse chow containing fructan. Culture-based enumerations are used to assess the inhibition of growth achieved within the microbiota upon inhibitor treatment. Compounds are also tested in humanized gnotobiotic mice, and impact on the relevant taxa determined using quantitative PCR and taxa-specific primers. Successful manipulation of the community is further validated using 16S-rRNA based enumeration to provide an in-depth view of how the presence of inhibitor influences changes in community composition.

Host responses and phenotypes associated with the altered microbiota are determined using a standard battery of tests. These tests utilize the Stanford Murine Phenotyping Core to complement assays that are implemented within the lab. These include (i) bomb calorimetry of feces to ascertain the energy extracted from the mouse chow by the host; (ii) gas chromatography-mass spectrometry based analysis of cecal glycans to establish the depletion of dietary substrates. Standard assays that can characterize changes in host innate and adaptive immune status, like serum cytokine levels and the relative representation of immunoglobulin isotypes, to microbiota manipulation are implemented. The epithelial response in the context of a conventional microbiota subjected to preferential expansion of the typically scarce Bifidobacteria (mediated by administration of fructan plus a Bacteroides fructan-utilization inhibitor) is characterized. Comparison of germ-free mouse responses upon exposure to the small molecule, but in the absence of a microbiota, provide an important control in identifying host responses that are mediated through the microbiota versus those that are a direct result of the small molecule treatment.

Choice of substrates and strains is guided by genomic, functional genomic, and genetic characterization of the relevant nutrient utilization systems and by the abundance of the strains in the microbiota before and after prebiotic treatment or in healthy and diseased states.

### Example 4

### Activator Screening

An alternative strategy for attaining improved specificity of prebiotics is to identify small molecules that enhance the machinery required for utilization of the specific prebiotic, for taxa that are exhibiting desired expansion. This activation of a nutrient utilization pathway within a specific taxon imposes a competitive advantage upon it and allows preferential expansion.

In the screening strategy and concept of the invention, a first screen is conducted to identify compounds that enhance growth of a targeted microorganism in defined medium, which medium may include a desired prebiotic as the sole carbon source. A simultaneous screen is conducted in glucose containing defined medium to identify the subset of compounds that shows specific activation. This strategy ensures that the activator is specific to the desired pathway by verifying growth in a counter-screen conducted using a carbon and energy source that is distinct from the prebiotic employed in the screen.

In an additional embodiment, a secondary screen is conducted on the sister species to identify which activators are species-specific versus those that activate, or enhance across species. By design, the latter strategy will select for compounds that activate across various species.

In a specific example, small molecules are arrayed into 384-well plates containing defined medium plus either fructan or glucose. Plates are transported into a 78-inch Coy anaerobic chamber that contains a liquid handler, plate reader, and 37° C. incubator. A saturated culture of Bifidobacteria is inoculated into each well, using an automated liquid dispenser (e.g. Multidrop). The screen is conducted at 37° C. in an anaerobic chamber, and growth measured (optical density at 600 nm) at appropriate intervals. Fructan-specific growth enhancing compounds are re-screened at eight different concentrations to establish a dose-response curve.

Compounds that enhance Bifidobacteria growth specifically on fructan, as defined above, are tested within gnotobiotic mice harboring model microbial communities composed of members of Bifidobacteria and Bacteroides. The small molecule is given orally to mice in conjunction with a custom mouse chow containing fructan. Culture-based enumerations are used to assess the inhibition of growth achieved within the microbiota upon enhancer treatment. Compounds are also tested in humanized gnotobiotic mice, and impact on the relevant taxa determined using quantitative PCR and taxa-specific primers. Successful manipulation of the community is further validated using 16S-rRNA based enumeration to provide an in-depth view of how the presence of inhibitor influences changes in community composition.

Host responses and phenotypes associated with the altered microbiota are determined using a standard battery of tests. These tests utilize the Stanford Murine Phenotyping Core to complement assays that are implemented within the lab. These include (i) bomb calorimetry of feces to ascertain the energy extracted from the mouse chow by the host; (ii) gas chromatography-mass spectrometry based analysis of cecal glycans to establish the depletion of dietary substrates. Standard assays that can characterize changes in host innate and adaptive immune status, like serum cytokine levels and the relative representation of immunoglobulin isotypes, to microbiota manipulation are implemented. The epithelial response in the context of a conventional microbiota subjected to preferential expansion of the typically scarce Bifidobacteria (mediated by administration of fructan plus a Bifidobacteria fructan-utilization enhancer) is characterized. Comparison of germ-free mouse responses upon exposure to the small molecule, but in the absence of a microbiota, provide an important control in identifying host responses that are mediated through the microbiota versus those that are a direct result of the small molecule treatment.

Choice of substrates and strains is guided by genomic, functional genomic, and genetic characterization of the relevant nutrient utilization systems and by the abundance of the strains in the microbiota before and after prebiotic treatment or in healthy and diseased states.

It is to be understood that this invention is not limited to the particular methodology, protocols, cell lines, animal species or genera, and reagents described, as such may vary. It is also to be understood that the terminology used herein is for the purpose of describing particular embodiments only, and is not intended to limit the scope of the present invention which will be limited only by the appended claims.

As used herein the singular forms “a”, “and”, and “the” include plural referents unless the context clearly dictates otherwise. All technical and scientific terms used herein have the same meaning as commonly understood to one of ordinary skill in the art to which this invention belongs unless clearly indicated otherwise.

The previous examples are put forth so as to provide those of ordinary skill in the art with a complete disclosure and description of how to make and use the subject invention, and are not intended to limit the scope of what is regarded as the invention. Efforts have been made to ensure accuracy with respect to the numbers used (e.g. amounts, temperature, concentrations, etc.) but some experimental errors and deviations should be allowed for. Unless otherwise indicated, parts are parts by weight, molecular weight is average molecular weight, temperature is in degrees centigrade; and pressure is at or near atmospheric.

1. A method for high-throughput screening of a candidate biological agent on the growth, substrate utilization, or phenotype of an anaerobic microorganism, the method comprising;
contacting a culture of an anaerobic microorganism with a candidate agent under assay conditions optimized to provide for highly controlled and reproducible conditions; and
determining the effect if said agent on the growth, substrate utilization, or phenotype of the anaerobic microorganism.

2. The method of claim 1, wherein said anaerobic microorganism is a species resident in a mammalian gut.

3. The method of claim 2, further comprising the step of repeating said contacting step with a second microbial strain.

4. The method of claim 2, wherein said assay conditions include culturing said microorganism in medium that has been formulated for improved stability by identifying a labile component, wherein the labile component is added to the medium shortly before inoculation with the microorganism.

5. The method of claim 2, wherein said assay conditions include optimizing the composition of anaerobic gases.

6. The method of claim 2, wherein said assay conditions include utilizing a synchronized microbial culture for inoculation.

7. The method of claim 2, wherein said assay conditions include utilizing media at a viscosity that is adjusted to maintain cell buoyancy throughout growth.

8. The method of claim 2, wherein said assay conditions include assay modifications to reduce edge effects.

9. A method for analyzing the effect of a candidate biologically active agent on a species of the microbiota, the method comprising:
contacting a first microbiota species culture with said candidate agent in the presence of a prebiotic compound that is a substrate for said microbiota species;
measuring growth parameters responsive to said prebiotic compound;
comparing said growth parameters in the presence of said candidate agent and in the absence of said candidate agent,
whereby an alteration of said growth parameters is indicative of the effect of said biologically active agent on said microbiota species.

10. The method of claim 9, further comprising the step of repeating said contacting step with a second, either evolutionarily- or functionally-related microbial strain.

11. The method of claim 10, wherein said prebiotic compound is a saccharide.

12. The method of claim 11, wherein said second microbiota strain and said first microbiota strain have a high degree of sequence similarity at a locus of interest that encodes a protein involved in utilization of said prebiotic compound.

13. The method of claim 12, wherein said locus of interest is a locus containing susC-like or susD-like genes.

14. The method of claim 9, wherein said contacting and culture is maintained in anaerobic conditions.

15. The method of claim 14, wherein said microbiota species is a Bacteroides or Firmicutes species.

16. The method of claim 14, further comprising the step of assessing the efficacy of said candidate agent in vivo by administering said agent to a non-human test animal in combination with said prebiotic, and determining the distribution of the microbiota following said administration.

17. The method of claim 16, wherein said non-human test animal comprises humanized microbiota.
